(19) United States
(12) Patent Application Publication
(10) Pub. No.: US 2019/0323038 A1
(43) Pub. Date: Oct. 24, 2019

(54) BIDIRECTIONAL TARGETING FOR GENOME EDITING

(71) Applicant: Montana State University, Bozeman, MT (US)

(72) Inventor: Blake A. Wiedenheft, Bozeman, MT (US)

(73) Assignee: Montana State Univesity, Bozeman, MT (US)

(21) Appl. No.: 16/310,387

(22) PCT Filed: Jun. 19, 2017
(86) PCT No.: PCT/US2017/038167

§ 371 Date: Dec. 14, 2018

Related U.S. Application Data
(60) Provisional application No. 62/351,507, filed on Jun. 17, 2016.

(51) Int. Cl. C12N 15/90; C12N 15/63; C12N 15/10; C12N 9/22

(52) U.S. Cl. CPC C12N 15/902; C12N 9/22; C12N 15/102; C12N 15/63

(57) ABSTRACT

Methods, systems and compositions for programmable gene modulation based on clustered regularly interspaced short palindromic repeats (CRISPRs) are provided. The methods comprise providing Cas3 nuclease and a pair of synthetic Type I CRISPR-Cas complexes to a cell comprising at least one target DNA sequence, for modulating the expression or function of the DNA sequence(s) in the cell to be edited, where the pair of Type I CRISPR-Cas complexes bind to sequences that flank the target DNA sequence to be edited.

### CROSS REFERENCE TO RELATED APPLICATIONS

This application claims the benefit of U.S. Provisional Application No. 62/351,507, filed Jun. 17, 2016, which is herein incorporated by reference in its entirety.

### STATEMENT OF GOVERNMENT SUPPORT

This invention was made with United States government support pursuant to grant GM108888, from the National Institutes of Health; the United States government has certain rights in the invention.

### FIELD

This disclosure relates to engineered, programmable, non-naturally occurring gene modulating systems, compositions of the system, and methods of carrying out genetic editing. In particular, methods, systems, and compositions are described that exploit bi-directional Type I CRISPR/Cas3 genetic editing.

### BACKGROUND

Clustered regularly interspaced short palindromic repeats (CRISPRs) and their associated genes (cas) are essential components of nucleic acid-based adaptive immune systems that are widespread in bacteria and archaea. CRISPR loci consist of a series of short repeats separated by non-repetitive spacer sequences, the spacer sequences of which are acquired from foreign genetic elements such as viruses and plasmids. Transcription of CRISPR loci generates a library of CRISPR-derived RNAs (crRNAs), containing sequences complementary to previously encountered invading nucleic acids. CRISPR-associated (Cas) proteins bind crRNAs, and the resultant ribonucleoprotein complex targets invading nucleic acids complementary to the crRNA guide. Targeted invading nucleic acids can be degraded by cis- or trans-acting nucleases.

Six main CRISPR system types (Types I to VI) and at least 24 distinct subtypes have been identified. All CRISPR systems use short CRISPR-derived RNAs (crRNAs) to target invading nucleic acid, and many of these nucleic acid targeting systems rely on sophisticated multi-subunit complexes. For example, the CRISPR-associated complex for antiviral defense (Cascade) is a Type I system composed of 11 protein subunits and a CRISPR-derived RNA (crRNA) complex that relies on complementary pairing between the crRNA-guide and a target nucleic acid sequence, which occurs over 32 nucleotides, or a portion thereof (FIGS. 1A & 1B). Type II systems, however, rely on a single protein (Cas9) and a 20 nucleotide sequence in recognizing invading DNA. Due to its relative simplicity, the Cas9 system has been used for commercial and research purposes in genetic engineering. Off-target nuclease activity has been detected, and these may limit the use of these tools for certain applications.

The Type I systems rely on a greater number of nucleotides for target DNA recognition, and employ a locking mechanism during target binding, which may be exploited as a gene modification device with enhanced specificity in target recognition compared to Cas9 systems. The complexity of the Type I CRISPR complex, the multiple reading frames and the delivery of these systems are hurdles to the use of Type I CRISPR complexes as a viable genome editing technology.

### SUMMARY

Cas3 is the trans-acting nuclease that is recruited to dsDNA-bound Cascade (or to other Type I CRISPR-Cas complexes) for target degradation (FIG. 1B). However, Cas3 cleaves only short regions of single-stranded DNA adjacent to dsDNA bound Cascade (or Csy). This is in stark contrast to the double-strand DNA cleavage exhibited by Cas9. In view of this discovery, there are enabled herein methods and systems for editing a double-stranded nucleic acid target sequence using any CRISPR systems that rely on Cas3, such as Cascade-Cas3 or Csy-Cas3, that uses paired Cascade-Cas3 complexes that bind to opposite strands of and at sites flanking the DNA target.

In examples of this system, Cascade complexes positioned within ˜200-400 base pairs of one another are used to delete a prescribed region of the target gene. This is illustrated schematically in FIG. 2.

Described herein are methods and systems that employ Cas3 with Type I CRISPR complexes, such as CRISPR-Cascade or CRISPR-CSY complexes, to carry out gene editing in a manner parallel to, for instance, Cas9-based CRISPR systems.

Thus, there is provided in a first embodiment a non-naturally occurring or engineered system for modifying a DNA (e.g., genomic) sequence in a cell (such as a eukaryotic or prokaryotic cell), the system comprising a first Type I CRISPR-Cas complex comprising a first guide RNA, a second Type I CRISPR-Cas complex comprising a second guide RNA that is different from the first guide RNA, and a Cas3 nuclease. In examples of this system, the first Type I CRISPR-Cas complex comprises a first guide RNA having a sequence selected to recognize a first target nucleotide sequence; and a plurality of Cas polypeptides and the second Type I CRISPR-Cas complex comprises a second (different from the first) guide RNA having a sequence selected to recognize a second target nucleotide sequence; and a plurality of Cas polypeptides, wherein the first and second target nucleotide sequences hybridize to opposite strands of the genomic DNA in the cell at positions that flank the genomic sequence to be modified.

Provided in another embodiment are non-naturally occurring cells (eukaryotic or prokaryotic cells) comprising any of the described systems for modifying a DNA sequence, or a vector or set of vectors expressing components of the system. It is contemplated that such cells in various examples will be prokaryotic (bacterial or archaeal), animal cells, plant cells, fungal cells, or algal cells. Optionally, such cells may contain a vector or set of vectors that comprise a nucleic acid sequence encoding at least one component of a Type I CRISPR-Cas complex of the system, which is codon optimized for expression in bacterial, archaea, or eukaryotic cells.

Another embodiment is a method for modifying a genomic sequence in a eukaryotic or prokaryotic cell, the method comprising contacting genomic DNA in the cell with: a first Type I CRISPR-Cas complex comprising a first guide RNA, a second Type I CRISPR-Cas complex comprising a second guide RNA, and a Cas3 nuclease, where the first and second guide RNAs each comprise a sequence that hybridizes to opposite strands of the genomic DNA in the cell at positions that flank the genomic sequence to be modified.

Also provided are methods for sequence-specific modification of a target nucleic acid sequence, the methods comprising targeting the nucleic acid sequence with the bi-directional, Type I CRISPR-Cas system described herein.

Yet other methods are methods for treating or preventing a disease in a subject in need of treatment or prevention, comprising administering to the subject a bi-directional, Type I CRISPR-Cas system as described herein.

Still additional provided embodiments are methods of producing a double-stranded break in a nucleic acid molecule in a cell, comprising introducing into the cell a first Type I CRISPR-Cas complex comprising a first crRNA comprising first target sequence, and a second Type I CRISPR-Cas complex comprising a second crRNA comprising second target sequence, where the first and second target sequences hybridize to opposite strands of genomic DNA in the cell at positions that flank the genomic sequence to be modified.

In any of the provided methods, it is contemplated that in some embodiments of the methods the first and second Type I CRISPR complexes are CRISPR-Cascade complexes (Type IE), or CRISPR-Csy complexes (Type IF), or any of the other Type I system that uses Cas3 for target degradation.

In the various examples of the provided embodiments of systems, cells, and methods, modifying the genomic sequence comprises deleting, inserting, or changing wild type genomic sequence. Optionally, this involves non-homologous end joining or homologous recombination at the double strand break generated by cleavage by the Cas3 nuclease.

Optionally, at least one component of one of the complexes, or the Cas3 nuclease, used in the provided systems, cells, and methods contains a nuclear localization sequence (NLS).

The foregoing and other features and advantages will become more apparent from the following detailed description of several embodiments, which proceeds with reference to the accompanying figures.

### BRIEF DESCRIPTION OF THE FIGURES

FIG. 1A schematically depicts the native Type IE CRISPR-Cascade operon from Escherichia coli.

FIG. 1B schematically depicts Cascade subunit assembly.

FIG. 2 is a schematic illustration of genome editing using bidirectional targeting of Cascade/Cas3. In the first illustration (1), Cascade binds to target sequences on opposite strands of the sequence to be edited (for instance, a gene to be inactivated; illustrated in some embodiments using a marker such as eGFP). (2) Cas3 introduces a single strand nick into the displaced (non-target) strand, and then (3) Cas3 degrades ˜200-400 bases of the displaced strand in a 3′-5′ direction. Use of two target sequences that flank the sequence to be edited permits the degradation of both strands of that sequence resulting in a region of deleted double-stranded DNA (dsDNA).

FIG. 3 is a schematic illustration of assemblage of Csy proteins into a ribonucleoprotein complex, showing crRNA (SEQ ID NO: 25) and DNA target strands (SEQ ID NOs: 26-27).

### SEQUENCE LISTING

The nucleic and amino acid sequences listed herein and provided in the accompanying Sequence Listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NO: 1 is the amino acid sequence of a Cse1 protein from Escherichia coli.

SEQ ID NO: 2 is the amino acid sequence of a Cse2 protein from Escherichia coli.

SEQ ID NO: 3 is the amino acid sequence of a Cas7 protein from Escherichia coli.

SEQ ID NO: 4 is the amino acid sequence of a Cas5 protein from Escherichia coli.

SEQ ID NO: 5 is the amino acid sequence of a Cas6 protein from Escherichia coli.

SEQ ID NO: 6 is the amino acid sequence of a Cas3 protein from Escherichia coli.

SEQ ID NO: 7 is the amino acid sequence of a Cse1 protein from Thermus thermophilus.

SEQ ID NO: 8 is the amino acid sequence of a Cse2 protein from Thermus thermophiles.

SEQ ID NO: 9 is the amino acid sequence of a Cas7 protein from Thermus thermophilus.

SEQ ID NO: 10 is the amino acid sequence of a Cas5 protein from Thermus thermophilus.

SEQ ID NO: 11 is the amino acid sequence of a Cas6 protein from Thermus thermophilus.

SEQ ID NO: 12 is the amino acid sequence of a Cas3 protein from Thermus thermophilus.

SEQ ID NO: 13 is the nucleic acid sequence of a Csy1 from Pseudomonas aeruginosa.

SEQ ID NO: 14 is the amino acid sequence of a Csy1 protein from Pseudomonas aeruginosa.

SEQ ID NO: 15 is the nucleic acid sequence of a Csy2 from Pseudomonas aeruginosa.

SEQ ID NO: 16 is the amino acid sequence of a Csy2 protein from Pseudomonas aeruginosa.

SEQ ID NO: 17 is the nucleic acid sequence of a Csy3 from Pseudomonas aeruginosa.

SEQ ID NO: 18 is the amino acid sequence of a Csy3 protein from Pseudomonas aeruginosa.

SEQ ID NO: 19 is the nucleic acid sequence of a Csy4 from Pseudomonas aeruginosa.

SEQ ID NO: 20 is the amino acid sequence of a Csy4 protein from Pseudomonas aeruginosa.

SEQ ID NO: 21 is the nucleic acid sequence of a Cas3 from Pseudomonas aeruginosa.

SEQ ID NO: 22 is the amino acid sequence of a Cas3 protein from Pseudomonas aeruginosa.

SEQ ID NO: 23 is the nucleic acid sequence of a Cas3 from Pseudomonas aeruginosa PA14.

SEQ ID NO: 24 is the amino acid sequence of a Cas1 protein from Pseudomonas aeruginosa.

SEQ ID NO: 25 is the nucleic acid sequence of a Csy crRNA.

SEQ ID NOs: 26 and 27 are the nucleic acid sequences of Csy DNA target (both strands).

### DETAILED DESCRIPTION

### 1. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

As used herein, the terms “polynucleotide,” “nucleic acid,” “oligonucleotide,” “oligomer,” “oligo,” or equivalent terms, refer to molecules that comprise a polymeric arrangement of nucleotide base monomers, where the sequence of monomers defines the polynucleotide. Polynucleotides can include polymers of deoxyribonucleotides to produce deoxyribonucleic acid (DNA), and polymers of ribonucleotides to produce ribonucleic acid (RNA). A polynucleotide can be single- or double-stranded. When single-stranded, the polynucleotide can correspond to the sense or antisense strand of a gene. A single-stranded polynucleotide can hybridize with a complementary portion of a target polynucleotide to form a duplex, which can be a homoduplex or a heteroduplex.

The length of a polynucleotide is not limited in any respect. Linkages between nucleotides can be internucleotide-type phosphodiester linkages, or any other type of linkage. A polynucleotide can be produced by biological means (e.g., enzymatically), either in vivo (in a cell) or in vitro (in a cell-free system). A polynucleotide can be chemically synthesized using enzyme-free systems. A polynucleotide can be enzymatically extendable or enzymatically non-extendable.

By convention, polynucleotides that are formed by 3′-5′ phosphodiester linkages (including naturally occurring polynucleotides) are said to have 5′-ends and 3′-ends because the nucleotide monomers that are incorporated into the polymer are joined in such a manner that the 5′ phosphate of one mononucleotide pentose ring is attached to the 3′ oxygen (hydroxyl) of its neighbor in one direction via the phosphodiester linkage. Thus, the 5′-end of a polynucleotide molecule generally has a free phosphate group at the 5′ position of the pentose ring of the nucleotide, while the 3′ end of the polynucleotide molecule has a free hydroxyl group at the 3′ position of the pentose ring. Within a polynucleotide molecule, a position that is oriented 5′ relative to another position is said to be located “upstream,” while a position that is 3′ to another position is said to be “downstream.” This terminology reflects the fact that polymerases proceed and extend a polynucleotide chain in a 5′ to 3′ fashion along the template strand. Unless denoted otherwise, whenever a polynucleotide sequence is represented, it will be understood that the nucleotides are in 5′ to 3′ orientation from left to right.

It is not intended that the term “polynucleotide” be limited to naturally occurring polynucleotide structures, naturally occurring nucleotides sequences, naturally occurring backbones, or naturally occurring internucleotide linkages. One familiar with the art knows well the wide variety of polynucleotide analogues, unnatural nucleotides, non-natural phosphodiester bond linkages, and internucleotide analogs that find use with the invention.

As used herein, the expressions “nucleotide sequence,” “sequence of a polynucleotide,” “nucleic acid sequence,” “polynucleotide sequence,” and equivalent or similar phrases refer to the order of nucleotide monomers in the nucleotide polymer. By convention, a nucleotide sequence is typically written in the 5′ to 3′ direction. Unless otherwise indicated, a particular polynucleotide sequence of the invention optionally encompasses complementary sequences, in addition to the sequence explicitly indicated.

As used herein, the term “guide sequence” refers to an RNA sequence that is part of the CRISPR complex and recognizes a target nucleic acid sequence. In some embodiments, the guide sequences are presented as DNA sequences which encode for the RNA sequences. In some embodiments, target recognition can occur through non-covalent interactions, including hydrogen bonding, recognition of a structural motif, nucleic acid sequence recognition, base pairing, the like, or any combination thereof. In other embodiments, target recognition can occur via covalent interactions.

As used herein, the term “gene” generally refers to a combination of polynucleotide elements, that when operatively linked in either a native or recombinant manner, provide some product or function. The term “gene” is to be interpreted broadly, and can encompass mRNA, cDNA, cRNA, and genomic DNA forms of a gene. In some uses, the term “gene” encompasses the transcribed sequences, including 5′ and 3′ untranslated regions (5′-UTR and 3′-UTR), exons, and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA (rRNA) genes and transfer RNA (tRNA) genes. In some aspects, the term “gene” includes not only the transcribed sequences, but in addition, also includes non-transcribed regions including upstream and downstream regulatory regions, enhancers and promoters. The term “gene” encompasses mRNA, cDNA, and genomic forms of a gene.

In some aspects, the genomic form or genomic clone of a gene includes the sequences of the transcribed mRNA as well as other non-transcribed sequences that lie outside of the transcript. The regulatory regions that lie outside the mRNA transcription unit are termed 5′ or 3′ flanking sequences. A functional genomic form of a gene typically contains regulatory elements necessary, and sometimes sufficient, for the regulation of transcription. The term “promoter” is generally used to describe a DNA region, typically but not exclusively 5′ of the site of transcription initiation, sufficient to confer accurate transcription initiation. In some aspects, a “promoter” also includes other cis-acting regulatory elements that are necessary for strong or elevated levels of transcription, or confer inducible transcription. In some embodiments, a promoter is constitutively active, while in alternative embodiments, the promoter is conditionally active (e.g., where transcription is initiated only under certain physiological conditions).

Generally, the term “regulatory element” refers to any cis-acting genetic element that controls some aspect of the expression of nucleic acid sequences. In some uses, the term “promoter” comprises essentially the minimal sequences required to initiate transcription. In some uses, the term “promoter” includes the sequences to start transcription, and in addition, also includes sequences that can upregulate or downregulate transcription, commonly termed “enhancer elements” and “repressor elements,” respectively.

Specific DNA regulatory elements, including promoters and enhancers, generally only function within a class of organisms. For example, regulatory elements from the bacterial genome generally do not function in eukaryotic organisms. However, regulatory elements from more closely related organisms frequently show cross functionality. For example, DNA regulatory elements from a particular mammalian organism, such as human, will most often function in other mammalian species, such as the mouse. Furthermore, in designing recombinant genes that will function across many species, there are consensus sequences for many types of regulatory elements that are known to function across species, e.g., in all mammalian cells, including mouse host cells and human host cells.

As used herein, the expressions “in operable combination,” “in operable order,” “operably linked,” “operatively linked,” “operatively joined,” and similar phrases, when used in reference to nucleic acids, refer to the operational linkage of nucleic acid sequences placed in functional relationships with each other. For example, an operatively linked promoter, enhancer elements, open reading frame, 5′ and 3′ UTR, and terminator sequences result in the accurate production of an RNA molecule. In some aspects, operatively linked nucleic acid elements result in the transcription of an open reading frame and ultimately the production of a polypeptide (that is, expression of the open reading frame).

As used herein, the term “genome” refers to the total genetic information or hereditary material possessed by an organism (including viruses), that is, the entire genetic complement of an organism or virus. The genome generally refers to all of the genetic material in an organism's chromosome(s), and in addition, extra-chromosomal genetic information that is stably transmitted to daughter cells (e.g., the mitochondrial genome). A genome can comprise RNA or DNA. A genome can be linear (mammals) or circular (bacterial). The genomic material typically resides on discrete units such as the chromosomes.

As used herein, a “polypeptide” is any polymer of amino acids (natural or unnatural, or a combination thereof), of any length, typically but not exclusively joined by covalent peptide bonds. A polypeptide can be from any source, e.g., a naturally occurring polypeptide, a polypeptide produced by recombinant molecular genetic techniques, a polypeptide from a cell, or a polypeptide produced enzymatically in a cell-free system. A polypeptide can also be produced using chemical (non-enzymatic) synthesis methods. A polypeptide is characterized by the amino acid sequence in the polymer. As used herein, the term “protein” is synonymous with polypeptide. The term “peptide” typically refers to a small polypeptide and typically is smaller than a protein. Unless otherwise stated, it is not intended that a polypeptide be limited by possessing or not possessing any particular biological activity.

As used herein, a “protein subunit,” “polypeptide subunit,” or “subunit” refers to a single protein molecule that assembles or coassembles with other protein or RNA molecules to form a protein or ribonucleoprotein (RNP) complex. Some naturally occurring proteins have a relatively small number of subunits and are therefore described as oligomeric, for example hemoglobin or DNA polymerase. Others may consist of a very large number of subunits and are therefore described as multimeric, for example microtubules and other cytoskeleton proteins. The subunits of a multimeric protein may be identical, homologous or totally dissimilar. For example, the CRISPR-Cascade ribonucleoprotein complex includes 11 subunits, which assemble around a crRNA. In some embodiments, the 11 protein subunits of Cascade include Cse1 (1 subunit), Cse2 (2 subunits), Cas7 (6 subunits), Cas5 (1 subunit), and Cas6 (1 subunit), as well as a 61-nucleotide crRNA. Similarly, the CRISPR-Csy ribonucleoprotein complex is a ˜350-kDa-ribonucleoprotein complex composed of 9 subunits of five functionally essential Cas proteins (one Csy1, one Csy2, six Csy3, and one Csy4) and a 60-nt crRNA-guide. These two ribonucleoprotein complexes are crRNA-guided DNA binding machines that recruit a trans-acting nuclease, Cas3 for target degradation.

As used herein, a “ribonucleoprotein complex” refers to a complex of protein and RNA. Examples of ribonucleoprotein (RNP) complexes include the ribosome, the enzyme telomerase, RNAse P, and small nuclear RNPs. In a specific embodiment, the ribonucleoprotein complex is CRISPR-Cas, CRISPR-Cascade, CRISPR-Csy or any CRISPR-RNA-guided complex that recruits Cas3 for target degradation.

As used herein, a protospacer-adjacent motif (PAM) is a short sequence motif immediately adjacent to the target of a CRISPR complex. PAMs are different in different organisms and PAM recognition is promiscuous in some systems.

As used herein, the expressions “codon utilization” or “codon bias” or “preferred codon utilization” or the like refers, in one aspect, to differences in the frequency of occurrence of any one codon from among the synonymous codons that encode for a single amino acid in protein-coding DNA or RNA (where many amino acids have the capacity to be encoded by more than one codon). In another aspect, “codon use bias” can also refer to differences between two species in the codon biases that each species shows. Different organisms often show different codon biases, where preferences for which codons from among the synonymous codons are favored in that organism's coding sequences.

As used herein, the terms “vector,” “vehicle,” “construct,” “template,” and “plasmid” are used in reference to any recombinant polynucleotide molecule that can be propagated and used to transfer nucleic acid segment(s) from one organism to another. Vectors generally comprise parts that mediate vector propagation and manipulation (e.g., one or more origin of replication, genes imparting drug or antibiotic resistance, a multiple cloning site, operably linked promoter/enhancer elements which enable the expression of a cloned gene, etc.). Vectors are generally recombinant nucleic acid molecules, often derived from bacteriophages or plant or animal viruses. Plasmids and cosmids refer to two such recombinant vectors.

A “cloning vector” or “shuttle vector” or “subcloning vector” contains operably linked parts that facilitate subcloning steps (e.g., a multiple cloning site containing multiple restriction endonuclease target sequences). A nucleic acid vector can be a linear molecule or in circular form, depending on type of vector or type of application. Some circular nucleic acid vectors can be intentionally linearized prior to delivery into a cell. Vectors can also serve as the template for polymerase chain reaction (PCR), to generate linear constructs, which may have additional sequences at their termini that are encoded by the primers used. Such constructs may also be delivered into a cell.

As used herein, the term “expression vector” refers to a recombinant vector comprising operably linked polynucleotide elements that facilitate and optimize expression of a desired gene (e.g., a gene that encodes a protein) in a particular host organism (e.g., a bacterial expression vector or mammalian expression vector). Polynucleotide sequences that facilitate gene expression can include, for example, promoters, enhancers, transcription termination sequences, and ribosome binding sites.

As used herein, the term “host cell” refers to any cell that contains a heterologous nucleic acid. The heterologous nucleic acid can be a vector, such as a shuttle vector or an expression vector, or linear DNA template, or in vitro transcribed RNA. In some aspects, the host cell is able to drive the expression of genes that are encoded on the vector. In some aspects, the host cell supports the replication and propagation of the vector. Host cells can be bacterial cells such as E. coli, animal cells, such as mammalian cells (e.g., human cells or mouse cells), or plant cells. When a suitable host cell is used to create a stably integrated cell line, that cell line can be used to create a complete transgenic organism.

Methods (that is, means) for delivering vectors/constructs or other nucleic acids (such as in vitro transcribed RNA) into host cells such as bacterial cells and mammalian cells are well known to one of ordinary skill in the art and are not provided in detail herein. Any method for nucleic acid delivery into a host cell finds use with the invention.

For example, methods for delivering vectors or other nucleic acid molecules into bacterial cells (termed “transformation”) such as Escherichia coli are routine, and include electroporation methods and transformation of E. coli cells that have been rendered competent by previous treatment with divalent cations such as CaCl₂.

Methods for delivering vectors, other nucleic acids (such as RNA), or RNPs into mammalian or plant cells in culture (termed transfection) are routine, and a number of transfection methods find use with the invention. These include but are not limited to calcium phosphate precipitation, electroporation, lipid-based methods (liposomes or lipoplexes) such as Transfectamine™ (Life Technologies™) and TransFectin™ (Bio-Rad Laboratories), cationic polymer transfections, for example using DEAE-dextran, direct nucleic acid injection, biolistic particle injection, and viral transduction using engineered viral carriers (termed “transduction,” using e.g., engineered herpes simplex virus, adenovirus, adeno-associated virus, vaccinia virus, Sindbis virus), and sonoporation. Any of these methods find use with the invention.

As used herein, the term “recombinant” in reference to a nucleic acid or polypeptide indicates that the material (e.g., a recombinant nucleic acid, gene, polynucleotide, polypeptide, etc.) has been altered by human intervention. Generally, the arrangement of parts of a recombinant molecule is not a native configuration, or the primary sequence of the recombinant polynucleotide or polypeptide has in some way been manipulated. A naturally occurring nucleotide sequence becomes a recombinant polynucleotide if it is removed from the native location from which it originated (e.g., a chromosome), or if it is transcribed from a recombinant DNA construct. A naturally occurring polypeptide sequence becomes a recombinant polypeptide if it is removed from the native location from which it originated, or if its native sequence is modified (e.g., insertion and/or deletion of amino acids). A gene open reading frame is a recombinant molecule if that nucleotide sequence has been removed from its natural context and cloned into any type of nucleic acid vector (even if that ORF has the same nucleotide sequence as the naturally occurring gene) or PCR template. Protocols and reagents to produce recombinant molecules, especially recombinant nucleic acids, are well known to one of ordinary skill in the art. In some embodiments, the term “recombinant cell line” refers to any cell line containing a recombinant nucleic acid, that is to say, a nucleic acid that is not native to that host cell.

As used herein, the terms “heterologous” or “exogenous” as applied to polynucleotides or polypeptides refers to molecules that have been rearranged or artificially supplied to a biological system and may not be in a native configuration (e.g., with respect to sequence, genomic position, or arrangement of parts) or are not native to that particular biological system. These terms indicate that the relevant material originated from a source other than the naturally occurring source or refers to molecules having a non-natural or non-native configuration, genetic location, or arrangement of parts. The terms “exogenous” and “heterologous” are sometimes used interchangeably with “recombinant.”

As used herein, the terms “native” or “endogenous” refer to molecules that are found in a naturally occurring biological system, cell, tissue, species, or chromosome under study as well as to sequences that are found within the specific biological system, cell, tissue, species, or chromosome being manipulated. A “native” or “endogenous” gene is generally a gene that does not include nucleotide sequences other than nucleotide sequences with which it is normally associated in nature (e.g., a nuclear chromosome, mitochondrial chromosome, or chloroplast chromosome). An endogenous gene, transcript, or polypeptide is encoded by its natural locus and is not artificially supplied to the cell.

As used herein, the terms “non-naturally occurring gene editing complex,” “engineered non-naturally occurring gene editing complex,” “non-naturally occurring complex,” and “non-naturally occurring CRISPR-Cascade (or -Csy) complex” refer to gene editing complexes that do not occur in nature. In some embodiments, the CRISPR associated proteins are Cascade proteins or Csy proteins. In some embodiments, the Type I CRISPR-Cas complexes used in the described methods and systems are concatenated or partially concatenated complexes, in which a plurality of subunits of the Type I CRISPR-Cas complex are tethered to each other, or the stoichiometry of the Type I CRISPR-Cas complex is modified, and/or the nucleotides in the crRNA are modified. In some embodiments, an engineered Type I CRISPR-Cas complex includes at least one subunit of the following proteins wherein two or more of the following subunits are tethered: (i) Cse1, (ii) Cse2, (iii) Cas7, (iv) Cas5, and (v) Cash; or (i) Csy1, (ii) Csy2, (iii) Csy3, and (iv) Csy4. In some embodiments, the length of the crRNA is modified. Thus, these non-natural complexes are composed of CRISPR associated proteins and crRNA, but these proteins and/or crRNA have been modified or are in an arrangement that does not occur in nature, and which results from the manipulation of man that occurs during the engineering of the complex.

As used herein, the terms “linker,” “linkage,” “tether,” “fused,” “joined,” and derivatives thereof refer to a means to connect subunits or to a connection between subunits. Accordingly, the terms include, but are not limited to, any compound, organic, inorganic, or a hybrid organic and inorganic compound, that connects, covalently or non-covalently, two subunits. “Linker,” “linkage,” “tether,” “fused,” and “joined” and derivatives thereof may be used interchangeably herein. By way of example, a nuclease (such as Cas3) can be linked to a Cas protein in a Type I CRISPR-Cas complex, such as a CRISPR-Cascade or CRISPR-Csy complex.

As used herein, the term “marker” most generally refers to a biological feature or trait that, when present in a cell (e.g., is expressed), results in an attribute or phenotype that visualizes or identifies the cell as containing that marker. A variety of marker types are commonly used and can be, for example, visual markers such as color development, e.g., lacZ complementation (β-galactosidase) or fluorescence, e.g., such as expression of green fluorescent protein (GFP) or GFP fusion proteins, red fluorescent protein (RFP), blue fluorescent protein (BFP), selectable markers, phenotypic markers (growth rate, cell morphology, colony color or colony morphology, temperature sensitivity), auxotrophic markers (growth requirements), antibiotic sensitivities and resistances, molecular markers such as biomolecules that are distinguishable by antigenic sensitivity (e.g., blood group antigens and histocompatibility markers), cell surface markers (for example H2KK), enzymatic markers, and nucleic acid markers, for example, restriction fragment length polymorphisms (RFLP), single nucleotide polymorphisms (SNP), and various other amplifiable genetic polymorphisms.

As used herein, the expression “selectable marker” or “screening marker” or “positive selection marker” refers to a marker that, when present in a cell, results in an attribute or phenotype that allows selection or segregation of those cells from other cells that do not express the selectable marker trait. A variety of genes are used as selectable markers, e.g., genes encoding drug resistance or auxotrophic rescue are widely known. For example, kanamycin (neomycin) resistance can be used as a trait to select bacteria that have taken up a plasmid carrying a gene encoding for bacterial kanamycin resistance (e.g., the enzyme neomycin phosphotransferase II). Non-transfected cells will eventually die off when the culture is treated with neomycin or similar antibiotic.

A similar mechanism can also be used to select for transfected mammalian cells containing a vector carrying a gene encoding for neomycin resistance (either one of two aminoglycoside phosphotransferase genes; the neo selectable marker). This selection process can be used to establish stably transfected mammalian cell lines. Geneticin (G418) is commonly used to select the mammalian cells that contain stably integrated copies of the transfected genetic material.

As used herein, the expression “negative selection” or “negative screening marker” refers to a marker that, when present (e.g., expressed, activated, or the like) allows identification of a cell that does not comprise a selected property or trait (e.g., as compared to a cell that does possess the property or trait).

A wide variety of positive and negative selectable markers are known for use in prokaryotes and eukaryotes, and selectable marker tools for plasmid selection in bacteria and mammalian cells are widely available. Bacterial selection systems include, for example but are not limited to, ampicillin resistance (beta-lactamase), chloramphenicol resistance, kanamycin resistance (aminoglycoside phosphotransferases), and tetracycline resistance. Mammalian selectable marker systems include, for example are but not limited to, neomycin/G418 (neomycin phosphotransferase II), methotrexate resistance (dihydrofolate reductase; DHFR), hygromycin-B resistance (hygromycin-B phosphotransferase), and blasticidin resistance (blasticidin S deaminase).

As used herein, the term “reporter” refers generally to a moiety, chemical compound, or other component that can be used to visualize, quantitate, or identify desired components of a system of interest. Reporters are commonly, but not exclusively, genes that encode reporter proteins. For example, a “reporter gene” is a gene that, when expressed in a cell, allows visualization or identification of that cell, or permits quantitation of expression of a recombinant gene. For example, a reporter gene can encode a protein, for example, an enzyme whose activity can be quantitated, for example, chloramphenicol acetyltransferase (CAT) or firefly luciferase protein. Reporters also include fluorescent proteins, for example, green fluorescent protein (GFP) or any of the recombinant variants of GFP, including enhanced GFP (EGFP), blue fluorescent proteins (BFP and derivatives), cyan fluorescent protein (CFP and other derivatives), yellow fluorescent protein (YFP and other derivatives) and red fluorescent protein (RFP and other derivatives).

As used herein, the term “tag” as used in protein tags refers generally to peptide sequences that are genetically fused to other protein open reading frames, thereby producing recombinant fusion proteins. Ideally, the fused tag does not interfere with the native biological activity or function of the larger protein to which it is fused. Protein tags are used for a variety of purposes, for example but not limited to, tags to facilitate purification, detection, or visualization of the fusion proteins. Some peptide tags are removable by chemical agents or by enzymatic means, such as by target-specific proteolysis (e.g., by TEV).

Depending on use, the terms “marker,” “reporter,” and “tag” may overlap in definition, where the same protein or polypeptide can be used as a marker, a reporter, or a tag in different applications. In some scenarios, a polypeptide may simultaneously function as a reporter and/or a tag and/or a marker, all in the same recombinant gene or protein.

As used herein, the term “prokaryote” refers to organisms belonging to the Kingdom Monera (also termed Procarya), generally distinguishable from eukaryotes by their unicellular organization, asexual reproduction by budding or fission, the lack of a membrane-bound nucleus or other membrane-bound organelles, a circular chromosome, the presence of operons, the absence of introns, message capping and poly-A mRNA, a distinguishing ribosomal structure, and other biochemical characteristics. Prokaryotes include subkingdoms Eubacteria (“true bacteria”) and Archaea (sometimes termed “archaebacterial”).

As used herein, the terms “bacteria” or “bacterial” refer to prokaryotic Eubacteria and are distinguishable from Archaea based on a number of well-defined morphological and biochemical criteria.

As used herein, the term “eukaryote” refers to organisms (typically multicellular organisms) belonging to the Kingdom Eucarya and are generally distinguishable from prokaryotes by the presence of a membrane-bound nucleus and other membrane-bound organelles, linear genetic material (that is, linear chromosomes), the absence of operons, the presence of introns, message capping and poly-A mRNA, a distinguishing ribosomal structure, and other biochemical characteristics.

As used herein, the terms “mammal” or “mammalian” refer to a group of eukaryotic organisms that are endothermic amniotes distinguishable from reptiles and birds by the possession of hair, three middle ear bones, mammary glands in females, a brain neocortex, and most giving birth to live young. The largest group of mammals, the placentals (Eutheria), has a placenta which feeds the offspring during pregnancy. The placentals include the orders Rodentia (including mice and rats) and primates (including humans).

A “subject” in the context of the present invention is preferably a eukaryotic organism, such as a fungus, algae, animal, or plant. Animals include for instance mammals, such as a human, non-human primate, mouse, rat, dog, cat, horse, or cow, but are not limited to these examples. Representative, non-limiting example plants include Arabidopsis; field crops (e.g., alfalfa, barley, bean, clover, corn, cotton, flax, lentils, maize, pea, rape/canola, rice, rye, safflower, sorghum, soybean, sunflower, tobacco, and wheat); vegetable crops (e.g., asparagus, beet, brassica generally, broccoli, Brussels sprouts, cabbage, carrot, cauliflower, celery, cucumber (cucurbits), eggplant, lettuce, mustard, onion, pepper, potato, pumpkin, radish, spinach, squash, taro, tomato, and zucchini); fruit and nut crops (e.g., almond, apple, apricot, banana, blackberry, blueberry, cacao, cassava, cherry, citrus, coconut, cranberry, date, hazelnut, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); tree woods and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose and rubber).

As used herein, the term “encode” refers broadly to any process whereby the information in a polymeric macromolecule is used to direct the production of a second molecule that is different from the first. The second molecule may have a chemical structure that is different from the chemical nature of the first molecule.

For example, in some aspects, the term “encode” describes the process of semi-conservative DNA replication, where one strand of a double-stranded DNA molecule is used as a template to encode a newly synthesized complementary sister strand by a DNA-dependent DNA polymerase. In other aspects, a DNA molecule can encode an RNA molecule (e.g., by the process of transcription that uses a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

As used herein, the term “derived from” refers to a process whereby a first component (e.g., a first molecule), or information from that first component, is used to isolate, derive, or make a different second component (e.g., a second molecule that is different from the first). For example, a codon-optimized CRISPR complex is derived from the corresponding wild type CRISPR complex.

As used herein, the expression “variant” refers to a first composition (e.g., a first molecule), that is related to a second composition (e.g., a second molecule, also termed a “parent” molecule). The variant molecule can be derived from, isolated from, based on, or homologous to the parent molecule.

As applied to polynucleotides, a variant molecule can have entire nucleotide sequence identity with the original parent molecule or, alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99%, or more identical in nucleotide sequence compared to the original nucleotide sequence. Polynucleotide variants also include polynucleotides comprising the entire parent polynucleotide and further comprise additional fused nucleotide sequences. Polynucleotide variants also include polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants include nucleotide sequences that contain minor, trivial, or inconsequential changes to the parent nucleotide sequence. For example, minor, trivial, or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence but have little or no impact on the biological activity of the polypeptide, and/or (iv) result in the substitution of an amino acid with a chemically similar amino acid. In the case where a polynucleotide does not encode for a protein (for example, a tRNA or a crRNA or a tracrRNA or a sgRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

As applied to proteins, a variant polypeptide can have entire amino acid sequence identity with the original parent polypeptide or, alternatively, can have less than 100% amino acid identity with the parent protein. For example, a variant of an amino acid sequence can be a second amino acid sequence that is at least 50%, 60%, 70%, 80%, 90%, 95%, 98%, 99%, or more identical in amino acid sequence compared to the original amino acid sequence.

Polypeptide variants include polypeptides comprising the entire parent polypeptide and further comprising additional fused amino acid sequences. Polypeptide variants also include polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants include polypeptides that contain minor, trivial, or inconsequential changes to the parent amino acid sequence. For example, minor, trivial, or inconsequential changes include amino acid changes (including substitutions, deletions, and insertions) that have little or no impact on the biological activity of the polypeptide and yield functionally identical polypeptides, including additions of non-functional peptide sequence. In other aspects, the variant polypeptides of the invention change the biological activity of the parent molecule.

In some aspects, polynucleotide or polypeptide variants of the invention can include variant molecules that alter, add, or delete a small percentage of the nucleotide or amino acid positions, for example, typically less than about 10%, less than about 5%, less than 4%, less than 2%, or less than 1%.

As used herein, the term “conservative substitutions” in a nucleotide or amino acid sequence refers to changes in the nucleotide sequence that either (i) do not result in any corresponding change in the amino acid sequence due to the redundancy of the triplet codon code, or (ii) result in a substitution of the original parent amino acid with an amino acid having a chemically similar structure. Conservative substitution tables providing functionally similar amino acids are well known in the art, where one amino acid residue is substituted for another amino acid residue having similar chemical properties (e.g., aromatic side chains or positively charged side chains) and therefore does not substantially change the functional properties of the resulting polypeptide molecule.

The following are groupings of natural amino acids that contain similar chemical properties, where substitution within a group is a “conservative” amino acid substitution. This grouping indicated below is not rigid, as these natural amino acids can be placed in different groupings when different functional properties are considered Amino acids having nonpolar and/or aliphatic side chains include: glycine, alanine, valine, leucine, isoleucine and proline. Amino acids having polar, uncharged side chains include: serine, threonine, cysteine, methionine, asparagine and glutamine. Amino acids having aromatic side chains include: phenylalanine, tyrosine and tryptophan. Amino acids having positively charged side chains include: lysine, arginine and histidine. Amino acids having negatively charged side chains include: aspartate and glutamate.

As used herein, the terms “identical” or “percent identity” in the context of two or more nucleic acids or polypeptides refer to two or more sequences or subsequences that are the same (“identical”) or have a specified percentage of amino acid residues or nucleotides that are identical (“percent identity”) when compared and aligned for maximum correspondence with a second molecule, as measured using a sequence comparison algorithm (e.g., by a BLAST alignment, or any other algorithm known to persons of skill), or, alternatively, by visual inspection.

The phrase “substantially identical” in the context of two nucleic acids or polypeptides refers to two or more sequences or subsequences that have at least about 60%, about 70%, about 80%, about 90%, about 90-95%, about 95%, about 98%, about 99%, or more nucleotide or amino acid residue identity, when compared and aligned for maximum correspondence using a sequence comparison algorithm or by visual inspection. Such “substantially identical” sequences are typically considered to be “homologous,” without reference to actual ancestry. Preferably, the “substantial identity” between nucleotides exists over a region of the polynucleotide at least about 50 nucleotides in length, at least about 100 nucleotides in length, at least about 200 nucleotides in length, at least about 300 nucleotides in length, or at least about 500 nucleotides in length, most preferably over their entire length of the polynucleotide. Preferably, the “substantial identity” between polypeptides exists over a region of the polypeptide at least about 50 amino acid residues in length, more preferably over a region of at least about 100 amino acid residues, and most preferably, the sequences are substantially identical over their entire length.

The phrase “sequence similarity” in the context of two polypeptides refers to the extent of relatedness between two or more sequences or subsequences. Such sequences will typically have some degree of amino acid sequence identity, and, in addition, where there exists amino acid non-identity, there is some percentage of substitutions within groups of functionally related amino acids. For example, substitution (misalignment) of a serine with a threonine in a polypeptide is sequence similarity (but not identity).

As used herein, the term “homologous” refers to two or more amino acid sequences when they are derived, naturally or artificially, from a common ancestral protein or amino acid sequence. Similarly, nucleotide sequences are homologous when they are derived, naturally or artificially, from a common ancestral nucleic acid. Homology in proteins is generally inferred from amino acid sequence identity and sequence similarity between two or more proteins. The precise percentage of identity and/or similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are generally available.

As used herein, the terms “portion,” “subsequence,” “segment,” or “fragment,” or similar terms refer to any portion of a larger sequence (e.g., a nucleotide subsequence or an amino acid subsequence) that is smaller than the complete sequence from which it was derived. The minimum length of a subsequence is generally not limited, except that a minimum length may be useful in view of its intended function. The subsequence can be derived from any portion of the parent molecule. In some aspects, the portion or subsequence retains a critical feature or biological activity of the larger molecule, or corresponds to a particular functional domain of the parent molecule, for example, the DNA-binding domain or the transcriptional activation domain. Portions of polynucleotides can be any length, for example, at least 5, 10, 15, 20, 25, 30, 40, 50, 75, 100, 150, 200, 300, or 500 or more nucleotides in length.

As used herein, the term “kit” is used in reference to a combination of articles that facilitate a process, method, assay, analysis, or manipulation of a sample. Kits can contain written instructions describing how to use the kit (e.g., instructions describing the methods of the present invention), chemical reagents or enzymes required for the method, primers and probes, as well as any other components.

“Therapeutic effect” as used herein refers to an effect on a disease or condition that is a measurable improvement in the progression, symptoms, or phenotype of the disease or condition. A “therapeutic treatment” or “therapeutically effective amount” provides a therapeutic effect in a subject. A therapeutic effect may be a partial improvement or may be a complete resolution of the disease or disorder. A therapeutic effect may also be an effect on a disease or condition as measured using a test system recognized in the art for the particular disease or condition. A therapeutic effect may also be a prophylactic effect, such that the disease or condition may be prevented, or such that symptoms of an underlying disease or condition may be prevented before they occur. For example, the systems, methods, compositions, and kits disclosed herein may be used to correct or improve a gene product such that the onset of a disease or condition, or an infection with an infectious agent, is prevented or delayed or ameliorated.

A “gene product” as used herein refers to a product of gene expression. In various embodiments, the gene product is a protein or enzyme; however, a gene product may also be RNA (e.g., when the gene codes for a non-protein product such as functional RNA).

As used herein, “wild-type polypeptide” or “wild-type protein” refers to a polypeptide encoded by a wild-type gene. A genetic locus can have more than one sequence or allele in a population of individuals, and the term “wild-type” encompasses all such naturally-occurring alleles that encode a gene product performing the normal function.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Thus, for example, reference to “a cell” includes combinations of two or more cells, or entire cultures of cells; reference to “a polynucleotide” includes, as a practical matter, many copies of that polynucleotide; and reference to “a polypeptide” can include multiple copies of that polypeptide. Similarly, the word “or” is intended to include “and” unless the context clearly indicates otherwise. Hence “comprising A or B” means including A, or B, or A and B.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### II. Overview of Several Embodiments

A first embodiment provided herein is a non-naturally occurring or engineered system for modifying a DNA (e.g., genomic) sequence in a cell (such as a eukaryotic cell), the system comprising a first Type I CRISPR-Cas complex comprising a first guide RNA, a second Type I CRISPR-Cas complex comprising a second guide RNA that is different from the first guide RNA, and a Cas3 nuclease. Examples of this system include a first Type I CRISPR-Cas complex comprising a first guide RNA having a sequence selected to recognize a first target nucleotide sequence; and a plurality of Cas polypeptides; and a second Type I CRISPR-Cas complex comprising a second (different from the first) guide RNA having a sequence selected to recognize a second target nucleotide sequence; and a plurality of Cas polypeptides; and a Cas3 nuclease, wherein the first and second target nucleotide sequences hybridize to opposite strands of the genomic DNA in the cell at positions that flank the genomic sequence to be modified. By way of example, the positions that flank the genomic sequence to be modified are in some instances at least about 100 bp apart but no more than about 1000 bp apart (for example, about 100-300 bp apart, about 200-400 bp apart, about 300-500 bp apart, about 400-600 bp apart, about 500-700 bp apart, about 600-800 bp apart, about 700-900 bp apart, or about 800-1000 bp apart). In other instances, the positions that flank the genomic sequence to be modified are about 200 bp to about 400 bp apart.

In the provided systems for modifying a DNA sequence in a cell, it is contemplated that the first and/or second Type I CRISPR-Cas may be a CRISPR-Cascade complex(es). In such examples, the plurality of Cas polypeptides in the CRISPR-Cascade complex(es) comprises Cas6, Cse1, Cse2, Cas7, and Cas5.

In additional embodiments of the provided systems for modifying a DNA sequence in a cell, it is contemplated that the first and/or second Type I CRISPR-Cas may be a CRISPR-Csy complex(es). In such examples, the plurality of Cas polypeptides in the CRISPR-Csy complex(es) comprises Csy1, Csy2, Csy3, and Csy4.

Also contemplated are embodiments in which the system includes two different types of Type I CRISPR-Cas complex. For instance, there may be one CRISPR-Cascade complex and one CRISPR-Csy complex. Combinations of other Type I complexes are also contemplated. In each pair provided with a system, the guide sequences are different and selected to hybridize to opposite strands of the genomic DNA in the cell at positions that flank the genomic sequence to be modified. By way of example, the positions that flank the genomic sequence to be modified are in some instances at least about 100 bp apart but no more than about 1000 bp apart (for example, about 100-300 bp apart, about 200-400 bp apart, about 300-500 bp apart, about 400-600 bp apart, about 500-700 bp apart, about 600-800 bp apart, about 700-900 bp apart, or about 800-1000 bp apart). In other instances, the positions that flank the genomic sequence to be modified are about 200 bp to about 400 bp apart.

The Cas3 nuclease of the system may be a separate protein or it may be tethered (attached) to a Cas polypeptide in the first or second complex. In certain instances, each of the first and second complexes in the system will have attached thereto a Cas3 nuclease.

In certain examples of the systems described herein, one or the other or both Type I CRISPR-Cas complexes contain two or more Cas polypeptides that are genetically fused. Such complexes may be referred to as (partially) concatenated complexes.

Provided in yet another embodiment are non-naturally occurring eukaryotic cells comprising any of the described systems for modifying a DNA sequence, or a vector or set of vectors expressing components of the system. It is contemplated that such eukaryotic cells in various examples will be animal cells, plant cells, fungal cells, or algal cells. Optionally, such eukaryotic cells may contain a vector or set of vectors that comprise a nucleic acid sequence encoding at least one component of a Type I CRISPR-Cas complex of the system, which is codon optimized for expression in bacterial, archaea, or eukaryotic cells.

Another embodiment is a method for modifying a genomic sequence in a eukaryotic cell, the method comprising contacting genomic DNA in the cell with: a first Type I CRISPR-Cas complex comprising a first guide RNA, a second Type I CRISPR-Cas complex comprising a second guide RNA, and a Cas3 nuclease, where the first and second guide RNAs each comprise a sequence that hybridizes to opposite strands of the genomic DNA in the cell at positions that flank the genomic sequence to be modified. By way of example, the positions that flank the genomic sequence to be modified are in some instances at least about 100 bp apart but no more than about 1000 bp apart (for example, about 100-300 bp apart, about 200-400 bp apart, about 300-500 bp apart, about 400-600 bp apart, about 500-700 bp apart, about 600-800 bp apart, about 700-900 bp apart, or about 800-1000 bp apart). In other instances, the positions that flank the genomic sequence to be modified are about 200 bp to about 400 bp apart.

In examples of the provided methods, contacting the genomic DNA with the first and/or second Type I CRISPR-Cas complex and Cas3 comprises any one or more of introducing individual protein or nucleic acid components of the complex or Cas3 directly into the cell, introducing the Type I CRISPR-Cas complex directly into the cell, expressing one or more nucleic acids encoding components of the Type I CRISPR-Cas complex or Cas3 in the cell, or a combination of two or more thereof. Optionally, introducing the Type I CRISPR-Cas complex directly into the cell comprises microinjection into the cell, or microinjection directly into the nucleus of a eukaryotic cell.

Also provided are methods for sequence-specific modification of a target nucleic acid sequence, the methods comprising targeting the nucleic acid sequence with the bi-directional, Type I CRISPR-Cas system described herein.

Yet other methods are methods for treating or preventing a disease in a subject in need of treatment or prevention, comprising administering to the subject a bi-directional, Type I CRISPR-Cas system as described herein.

Still another provided embodiment is methods of producing a double-stranded break in a nucleic acid molecule in a cell, comprising introducing into the cell a first Type I CRISPR-Cas complex comprising a first crRNA comprising first target sequence, and a second Type I CRISPR-Cas complex comprising a second crRNA comprising second target sequence, where the first and second target sequences hybridize to opposite strands of genomic DNA in the cell at positions that flank the genomic sequence to be modified. By way of example, the positions that flank the genomic sequence to be modified are in some instances at least about 100 bp apart but no more than about 1000 bp apart (for example, about 100-300 bp apart, about 200-400 bp apart, about 300-500 bp apart, about 400-600 bp apart, about 500-700 bp apart, about 600-800 bp apart, about 700-900 bp apart, or about 800-1000 bp apart). In other instances, the positions that flank the genomic sequence to be modified are about 200 bp to about 400 bp apart.

In any of the provided methods, it is contemplated that in some embodiments of the methods the first and second Type I CRISPR complexes are CRISPR-Cascade complexes (Type IE), or CRISPR-Csy complexes (Type IF), or any of the other Type I system that uses Cas3 for target degradation.

In the various examples of the provided embodiments of systems, cells, and methods, modifying the genomic sequence comprises deleting, inserting, or changing wild type genomic sequence through homologous recombination at the double strand break generated by cleavage by the Cas3 nuclease. In other examples, modifying the genomic sequence comprises homologous recombination at the double strand break generated by cleavage by the Cas3 nuclease.

Optionally, at least one component of one of the complexes, or the Cas3 nuclease, used in the provided systems, cells, and methods contains a nuclear localization sequence (NLS).

III. CRISPR Gene Editing Systems

Genetic engineering through genome editing—the ability to insert, replace, and remove DNA—can confer disease resistance, remove malignant DNA, or improve crop production, among many other applications.

CRISPR is an RNA-guided adaptive immune mechanism by which bacteria and archaea resist infection from invading viruses and plasmids. Foreign genetic material from a virus or a plasmid is acquired by and stored in a CRISPR complex, and this information is used to recognize and degrade complementary nucleic acids upon subsequent invasion. Efficient detection of invading DNA relies on complementary base pairing between the DNA target and the crRNA-guide sequence, in addition to recognition of a short sequence motif immediately adjacent to the target (that is, a protospacer-adjacent motif (PAM)). This target nucleotide recognition mechanism allows for CRISPR technology to be adapted for genome editing.

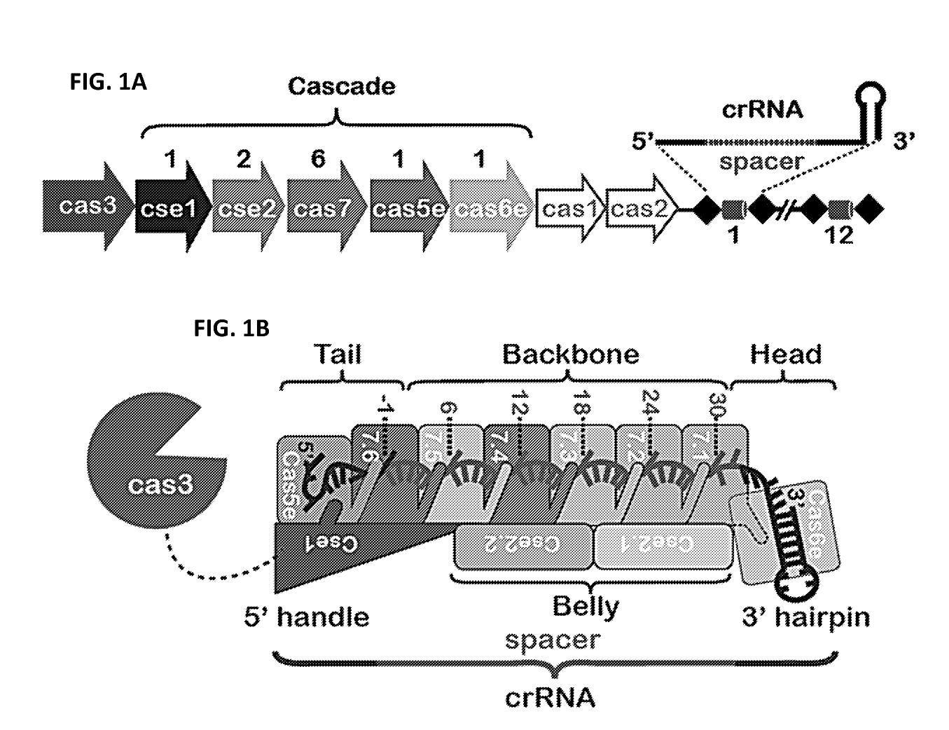

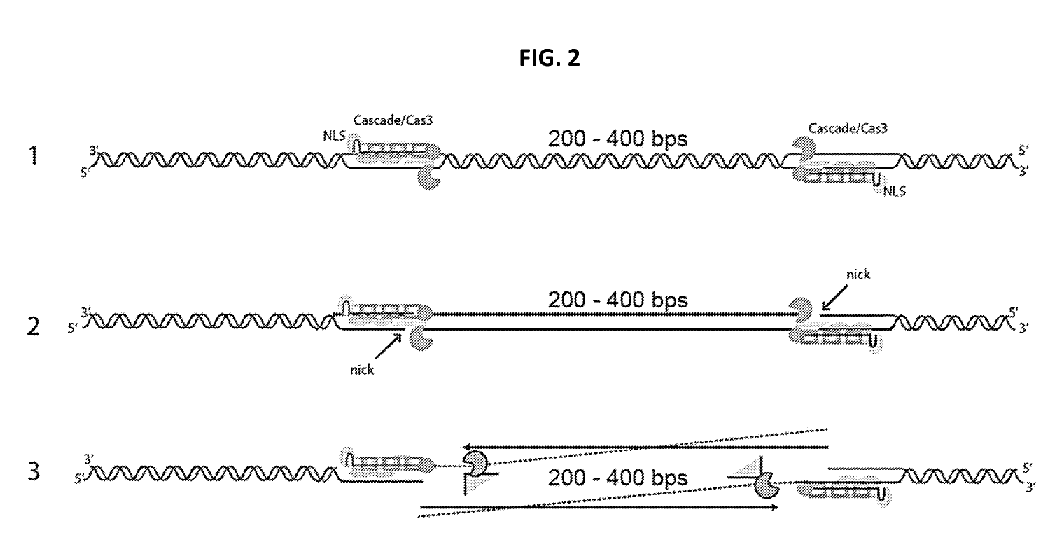

Phylogenetic and functional studies have identified six main CRISPR-system Types (I-VI) and 24 subtypes (including IA-F, IIA-C, IIIA-B). The Type IE system from Escherichia coli K12 consists of a CRISPR locus and eight cas genes (FIG. 1A). Five of the cas genes in this system encode for proteins that assemble with the crRNA into a large complex called Cascade (CRISPR-associated complex for antiviral defense). Efficient detection of invading DNA relies on complementary base pairing between the DNA target and crRNA-guide sequence, as well as recognition of a short sequence motif immediately adjacent to the target called a PAM. Target recognition by Cascade triggers a conformational change and recruits a transacting nuclease-helicase (Cas3) that is required for destruction of invading target.

The Type II Cas9 CRISPR system has been used for gene editing in eukaryotic systems. Cas9 is a single protein system that can be programmed with guide RNA to target any complementary nucleic acid sequence flanked by a short PAM sequence motif (WO 2014/093701 A1). The simplicity of the Cas9 system explains its widespread use in programmable genetic engineering in microorganisms, cell lines, plants, and animals (Wilkinson & Wiedenheft, F1000Prime Reports; 6:3, eCollection 2014; doi: 10.12703/P6-3).

The guide sequence of Cas9, however, tolerates mismatches between the guide sequence and the DNA-target sequence, and off-target effects (that is, cleavage at non-target sequences) are prevalent (Cho et al., Genome Res. 24(1):132-141, 2014; Fu et al., Nat. Biotechnol. 31(9):822-826, 2013; Hsu et al., Nat. Biotechnol. 31(9):827-832, 2013). Off-target cleavage occurs when the guide RNA targets Cas9 to a sequence that has one or more bases different from the guide sequence. Flexibility in target recognition is undesirable due to the potential for off-target cleavage, which limits the use of Cas9-derived tools as gene modification devices were accuracy is critical. Mutations of Cas9 have been performed in attempt to decrease off-target effects (WO 2014/093701 A1). Target recognition, however, still relies on recognition by a relatively small number of nucleotides.

For experimental and commercial applications (gene targeting, crop engineering, therapeutic applications, and so forth, as non-limiting examples), improving the specificity gene targeting and reducing the likelihood of off-target modification is critical. Some of the multi-subunit crRNA-guided Type I complexes (e.g. Cascade and Csy) have longer guide sequences that can be exploited to increase the accuracy of target recognition, and these sophisticated systems can be used as gene modification devices.

The Type I CRISPR-Cas systems offer a promising alternative to the Cas9 system. In contrast to Cas9, in which 20 nucleotides participate in target recognition (Sternberg et al., Nature 507(7490):62-67, 2014), recognition of target DNA by Type I CRISPR-Cas proceeds through interactions with a crRNA-guide sequence that includes about 32 nucleotides (Mulepati et al., Science 345(6203):1479-84, 2014; Jackson, et al., Science 345(6203):1473-9, 2014; Semenova, et al., PNAS 108(25):10098-103, 2011; and Brouns et al., Science 321(5891):960-964, 2008). Recent structural and biochemical studies explain which of the nucleotides in the guide are involved in base pairing with the target (Mulepati et al., Science 345(6203):1479-84, 2014; and Jackson et al., Science 345(6203):1473-9, 2014). The greater number of nucleotides participating in target recognition in Type I CRISPR-Cas complex systems has the ability to increase selectivity and, thus, reduce off-target effects.

Cascade is a Type I CRISPR system that consists of a CRISPR locus, eight Cas genes, and short CRISPR-derived RNAs (crRNAs) (Jackson et al., Science. 345(6203):1473-9, 2014). Five of the Cas genes in this system encode for proteins (Cse1, Cse2, Cas7, Cas5e, and Cas6e) that assemble with crRNA to form a 405 kDa multi-subunit surveillance complex called Cascade (CRISPR-associated complex for antiviral defense) (Brouns et al., Science. 321(5891):960-964, 2008).

Cascade is composed of 11 protein subunits and a 61-nucleotide crRNA, which assemble into a sea-horse-shaped architecture that binds double-stranded DNA targets complementary to the crRNA-guide sequence (Jackson et al., Science 345(6203):1473-9, 2014). Nine of the eleven Cas proteins make direct contact with the crRNA, and eight of the nine RNA-binding proteins contain a modified RNA Recognition Motif (RRM) (Jackson et al., Science. 345(6203):1473-9, 2014). The 5′ and 3′ ends of the crRNA are derived from the repeat region of the CRISPR RNA and are bound at opposite ends of the sea-horse-shaped complex. Cas6e binds the 3′ end of the crRNA at the head of the complex, while the 5′ end of the crRNA is sandwiched between three protein subunits (Cas5, Cas7.6, and Cse1) in the tail of the complex. The head and tail of the complex are connected along the belly by two Cse2 subunits and by a helical backbone composed of six Cas7 proteins (Cas7.1—Cas7.6). This assembly creates an interwoven ribonucleoprotein structure that kinks the crRNA at 6-nt intervals.

Upon target recognition by Cascade, a conformational change is induced and a transacting nuclease-helicase (e.g. Cas3) is recruited, which transacting nuclease-helicase is required for destruction of invading target DNA (Hochstrasser et al., PNAS 111(18):6618-6623, 2014; Wiedenheft et al., Nature 477:486-489, 2011; Westra et al., Mol. Cell 46(5):595-605, 2012; Jackson et al., Current Opinion in Structural Biology 24:106-114, 2014; Westra et al., Annu. Rev. Genet 46:311-339, 2012; Rollins et al., PNAS doi:10.1073/pnas.1616395114, 2017).

One Type I CRISPR-mediated adaptive immune system is the Csy complex. A representative Csy complex is from Pseudomonas aeruginosa as depicted in SEQ ID NOs: β-24. A representative Csy complex is from Vibrio phage ICP1 (Seed et al., Mol Biol. 8 Feb. 2011; 2(1): e00334-10). It is recognized that Csy peptides can include one or more of the mutations described in the literature, including but not limited to the functional mutations described in: Cady & O'Toole (J. Bacteriology 193(14):3433-3445, 2011). Thus, in some embodiments, the systems and methods disclosed herein can be used with a protein having double-stranded nuclease activity, a protein that acts as a single-stranded nickase, or mutant proteins with modified nuclease activity.

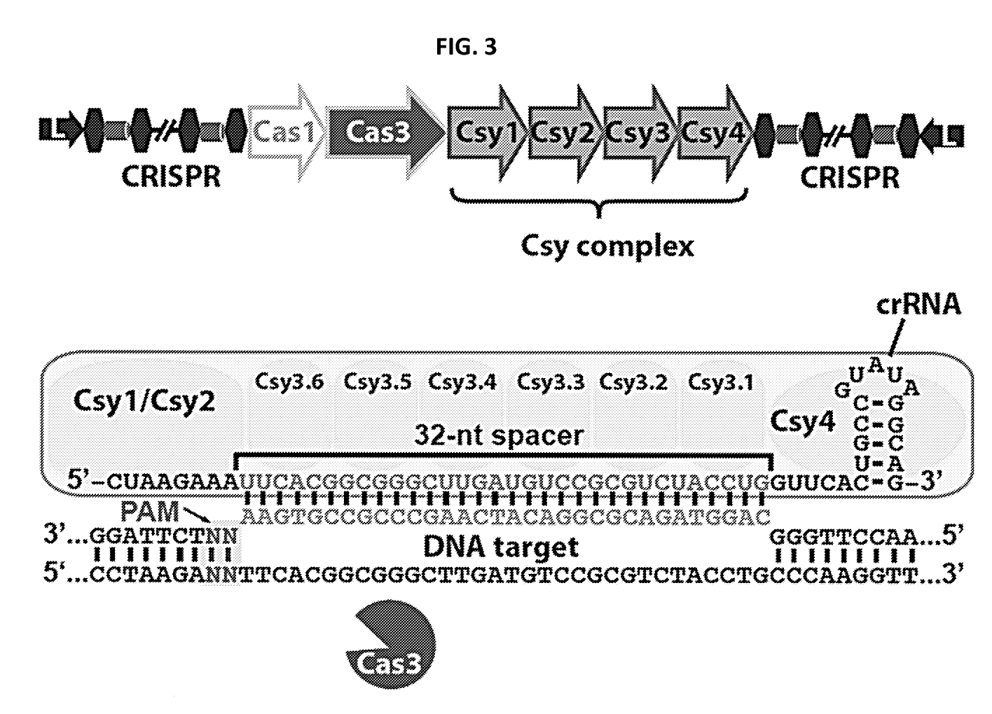

The Type I-F CRISPR-mediated adaptive immune system consists of two CRISPR loci, six cas genes (cas1, cas3, csy1, csy2, csy3, and csy4), and crRNA (Cady et al., J. Bacteriol 2012; 194: 5728-5738). Shown in FIG. 3 is the Csy complex from P. aeruginosa (PA14). Two CRISPR loci flank the set of cas genes (cas1, cas3, csy1, csy2, csy3, and csy4). Each CRISPR consists of a series of short repeats (black hexagons) that are separated by unique spacer sequences (cylinders). Each repeat is separated by unique spacer sequences (dashed black line). Four of the cas genes encode proteins (csy1, csy2, csy3, and csy4) that assemble with crRNA to form a 350-kDa ribonucleoprotein complex called Csy for surveillance of foreign DNA (Wiedenheft et al., Proc. Natl. Acad. Sci. USA; 108:10092-10097, 2011). The Csy ribonucleoprotein complex includes nine protein subunits with the following stoichiometry: one Csy1; one Csy2, six Csy3, one Csy4, and crRNA (Wiedenheft et al., Proc. Natl. Acad. Sci. USA; 108:10092-10097, 2011).

Target recognition by CRISPR-Csy is initiated by detection of a double-stranded PAM consisting of two consecutive guanine-cytosine base pairs (G-C/G-C) located adjacent to the complementary DNA target (Rollins et al., Nucl. Acids Res. 8 Feb. 2015; published online at doi: 10.1093/nar/gkv094). The CRISPR-Csy crRNA includes a portion near the 5′ that is complementary to a target nucleic acid. After recognition of the PAM, complementary base pairing between the crRNA and the target DNA recruits a nuclease protein to the target DNA. Once bound to a target, the nuclease domain cleaves the non-complementary strand of the double-stranded DNA (dsDNA) target sequence.

Additional information about crRNA-guided surveillance complex systems for gene editing can be found in the following documents, which are incorporated by reference in their entirety: U.S. Application Publication No. 2010/0076057 (Sontheimer et al., TARGET DNA INTERFERENCE WITH crRNA); U.S. Application Publication No. 2014/0179006 (Feng, CRISPR-CAS COMPONENT SYSTEMS, METHODS AND COMPOSITIONS FOR SEQUENCE MANIPULATION); U.S. Application Publication No. 2014/0294773 (Brouns et al., MODIFIED CASCADE RIBONUCLEOPROTEINS AND USES THEREOF); and Sorek et al., Annu. Rev. Biochem. 82:273-266, 2013.

### IV. Bi-Directional Targeting of CRISPR-Cas/Cas3

With the discovery that Cas3 nuclease cleaves only short regions of single-stranded DNA adjacent to dsDNA bound Cascade complexes, there is described herein for the first time a bi-directional system for gene editing that exploits two Type I CRISPR-Cas complexes, each loaded with a different RNA guide. By designing specific crRNA-guide sequences so that the first and second Type I CRISPR-Cas complex in the system bind to (associate with) opposite strands of a DNA molecule (for instance, genomic DNA) and on either side (that is, flanking) of the sequence to be modified, it is now possible to intentionally and specifically delete or otherwise edit the DNA sequence therebetween.

Methods are described for gene editing, which involve providing the first and second (NLS-tagged) Type I CRISPR-Cas complex (with their associated guide RNAs), along with a Cas3 nuclease (either separately NLS-tagged, or attached to a protein of the NLS-tagged Type I CRISPR-Cas complex), to a eukaryotic cell. These methods permit deletion of the region of DNA between the sites homologous to the guide RNA, through action of the Cas3 nuclease; by degrading one strand in each direction, the result is a double strand break. Notably, the first and second Type I CRISPR-Cas complex used in the systems described herein need not be the same; for instance, one Cascade (Type IE) and one Csy (Type IF) complex may be used in combination with each other or any other CRISPR system.

The positions in the DNA to which the guide RNAs bind are selected to be at least about 100 bps apart and up to about 500 bps apart, or in some embodiments 200-400 bps apart (for example, about 100-500 bp apart, about 100-300 bp apart, about 200-400 bp apart, or about 300-500 bp apart), thus permitting the processivity of the Cas3 nuclease to generate a double strand gap in the DNA. The target positions in the DNA to be modified (analogous to protospacers in a viral genome) are selected from regions that are flanked by PAMs, in order to enable recognition by the Type I CRISPR-Cas complex. Selection of an appropriate PAM sequence is within the skill of the ordinary skilled artisan, and is matched to the specific Type I CRISPR-Cas complex(s) that are being employed in the system (since the sequence of the PAM must be cognate to protein(s) in the complex). PAM are short and occur by chance at high frequency in a genome, thus protospacer selection is rarely limited by the requirement for a PAM.

Upon generation of the double strand break in the DNA molecule to be modified, the described system then harnesses the cell's own DNA repair mechanism(s) to complete the editing and close the gap. In some embodiments, this occurs through non-homologous end joining (which will result in deletion of some or all of the DNA sequence between the position at which the two complexes bound and the Cas3 nuclease initiated cleavage) and homologous repair, as described herein. In the instance of homologous repair, a repair template is provided in trans, as part of the system.

### V. Expression and Purification

In some embodiments, the present invention teaches methods and compositions of vectors, constructs, and nucleic acid sequences encoding for the non-natural, engineered gene editing complexes of the present invention. In some embodiments, the present invention teaches plasmids for transgenic or transient expression of the Type I CRISPR-Cas proteins. In some embodiments the present invention teaches a plasmid encoding a chimeric Type I CRISPR-Cas protein comprising in-frame sequences for protein fusions of one or more of the other proteins described herein, including, but not limited to a Type I CRISPR-Cas protein, a nuclease, a linker, and a nuclear localization sequence (NLS).

In some embodiments the plasmids and vectors of the present invention will encode for the proteins of the present invention and also encode the guide RNA of the present invention. In other embodiments, the different components of the engineered complex can be encoded in two or more distinct plasmids.

In some embodiments the plasmids of the present invention can be used across multiple species. In other embodiments, the plasmids of the present invention are tailored to the organism being transformed. In some embodiments, the sequences of the present invention will be codon-optimized to express in the organism whose genes are being edited. Persons having skill in the art will recognize the importance of using promoters providing adequate expression for gene editing. In some embodiments, the plasmids for different species will require different promoters.

In some embodiments, the plasmids and vectors of the present invention are selectively expressed in the cells of interest. Thus in some embodiments, the present application teaches the use of ectopic promoters, tissue-specific promoters, developmentally-regulated promoters, or inducible promoters. In some embodiments, the present invention also teaches the use of terminator sequences.

In other embodiments, a portion, or the entire complex(es) of the present technology, or the entire set of components of a bi-directional gene editing system, can be delivered directly to cells (e.g., through microinjection). Thus in some embodiments, the present invention teaches the expression and purification of the polypeptides and nucleic acids of the present invention. Persons having skill in the art will recognize the many ways to purify protein and nucleic acids. In some embodiments, the polypeptides can be expressed via inducible or constitutive protein production systems such as the bacterial system, yeast system, plant cell system, or animal cell systems. In some embodiments, the present invention also teaches the purification of proteins and or polypeptides via affinity tags, or custom antibody purifications. In other embodiments, the present invention also teaches methods of chemical synthesis for polynucleotides.

In some embodiments, the present invention teaches the use of transformation of the plasmids and vectors disclosed herein. Persons having skill in the art will recognize that the plasmids of the present invention can be transformed into cells through any known system. For example, in some embodiments, the present invention teaches transformation by particle bombardment, chemical transformation, agrobacterium transformation, nano-spike transformation, and virus transformation.

### Duration of Expression

In some aspects, the delivery vehicle for the guide sequence is selected such that the guide sequence is expressed in the target tissue for at least 1 week. However, longer expression will be desired in some embodiments, such as expression in the target tissue for at least 2 weeks, or at least 3 weeks, or at least 4 weeks, or at least 5 weeks, or at least 6 weeks, or at least 7 weeks, or at least 8 weeks, or for at least 2 months, at least 3 months, at least 4 months, at least 6 months, at least 8 months, at least 10 months, at least 12 months, at least 18 months, at least 24 months, or more. In some embodiments, the length of time of expression of the guide sequence provides a window in which an editing system is provided to the cells to effect the nucleic acid modification.

In some embodiments, the delivery systems, compositions, methods, and kits provided herein provide transient expression of the nucleic acid editing system (e.g., Cascade or Csy) in the target cell. In some embodiments, such transient expression helps to minimize off-target effects and/or immunogenicity. For example, in one embodiment, the delivery systems, compositions, methods, and kits provided herein provide expression of the nucleic acid editing system such as Type I CRISPR-Cas (e.g., Cascade or Csy) in a cell for about two weeks or less, or for about 1 week or less. In some embodiments, the compositions and delivery systems provided herein provide expression of the Type I CRISPR-Cas nucleic acid editing system in a cell for about 1 day to about 5 days, or for about 1 day to about 3 days.

The timing and type of expression of the Type I CRISPR-Cas guide sequence and/or nucleic acid editing system (such as Cascade or Csy) can be varied, such as through tissue-specific promoters, constitutive promoters or inducible promoters. As used herein, an inducible promoter is any promoter whose activity is regulated upon the application of an agent, such as a small molecule, to the cell. For example, inducible promoters include tetracycline-regulatable or doxycycline-regulatable promoters, carbohydrate-inducible or galactose-inducible promoters, isopropyl-beta-D-thiogalactopyranoside (IPTG)-regulated promoters, heat-shock promoters, and steroid-regulated promoters. In certain embodiments, the nucleic acid editing system and/or guide sequence is expressed from a tissue specific promoter, e.g., a promoter that is active in the target tissue more than some other tissues. For example, depending on the target tissue, the promoter is a tissue specific promoter that is expressed in one or more of liver, heart, lung, skeletal muscle, CNS, endothelial cells, stem cell, blood cell or blood cell precursor, and immune cells. Exemplary promoters include RNA III polymerase promoters, and viral promoters such as U6, CMV, SV40, EF-1α, Ubc, and PGK promoters, or derivatives thereof having comparable promoter strength. Other promoters can be selected and/or designed based on publicly available information (see, for example, the mammalian promoter database at mpromdb.wistar.upenn.edu). These and other promoters, expression control elements (e.g., enhancers), and constructs that can be used are described, for example, in U.S. Pat. No. 8,697,359, which is hereby incorporated by reference in its entirety.

The duration of expression of the nucleic acid editing system and/or guide sequence can be determined in a suitable cell line that is indicative of expression in the target tissue, and/or where the promoter of choice is expressed in a manner that is comparable with the target tissue. For example, where the target tissue is liver, the duration of expression of the nucleic acid editing system and/or guide sequence may be determined in hepatocyte cell culture such as HuH-7 or transformed primary human hepatocytes. Alternatively, Human Embryonic Kidney 293T cells may be used to quantify duration of expression. Expression can be measured by, for example, immunohistochemistry, RT-PCR, or flow cytometry. In some embodiments, a Type I CRISPR-Cas guide sequences or nucleic acid editing system (such as Cascade or Csy, for example) can be expressed with a suitable tag (e.g., HA tag) to monitor expression with commercially available antibodies. In some embodiments, the expression of the nucleic acid editing system and/or guide sequence and/or the efficiency of target nucleotide modification can be detected or monitored using reporter genes, reporter sequences, epitope tags, and/or expression tags. A “reporter gene” or “reporter sequence” or “epitope tag” or “expression tag” refers to any sequence that produces a product that is readily measured. Reporter genes include, for example, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG-tag, His, myc, Tap, HA or any detectable amino acid sequence. “Expression tags” include sequences that encode reporters that may be operably linked to a desired gene sequence in order to monitor expression of the gene of interest.

Other exemplary cell lines for which expression of the guide sequence(s) or nucleic acid editing system constructs may be quantified include: C8161, CCRF-CEM, MOLT, mIMCD-3, NHDF, HeLa-S3, Huh1, Huh4, Huh7, HUVEC, HASMC, HEKn, HEKa, MiaPaCell, Panel, PC-3, TF1, CTLL-2, C1R, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-MeI 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR.

### Delivery Systems

The efficient delivery of nucleic acid editing systems, including the bi-directional Type I CRISPR-Cas complex system, provide for safer and more effective delivery systems, which are especially useful in the clinical setting. Representative delivery systems herein disclose methods and compositions containing viral and/or non-viral vectors to deliver nucleic acid editing systems, particularly, Type I CRISPR-Cas complex system, and optionally an editing template to edit genes in cells. While gene editing is particularly useful in vivo, in some embodiments, the cell targeted for gene editing may be in vitro, ex vivo, or in vivo.

In some embodiments persons having skill in the art will recognize viral vectors or plasmids for gene expression can be used to deliver the engineered contiguous and/or extended Type I CRISPR-Cas complexes disclosed herein. Virus-like particles (VLP) can be used to encapsulate ribonucleoprotein complexes or recombinant expression, and purified ribonucleoprotein complexes disclosed herein can be purified and delivered to cells via electroporation or injection.

Delivery vehicles provided herein may be viral vectors or non-viral vectors, or RNA conjugates. In some embodiments, the guide sequence and the nucleic acid editing system are provided in the same type of delivery vehicle, wherein the delivery vehicle is a viral vector or a non-viral vector. In other embodiments, the guide sequence is provided in a viral vector, and the nucleic acid editing system is provided in a non-viral vector. In still other embodiments, the one or more guide sequence is provided in a non-viral vector and the nucleic acid editing system is provided in a viral vector. In some embodiments, the guide sequence is provided in an RNA conjugate.

Any vector systems may be used, including, but not limited to, plasmid vectors, linear constructs, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more CRISPR-Cas encoding sequences and/or additional nucleic acids as appropriate. Thus, when one or more Type I CRISPR-Cas proteins and/or guide sequence as described herein are introduced into the cell, and additional DNAs as appropriate, they may be carried on the same vector or on different vectors. When multiple constructs are used, each vector may comprise a sequence encoding one or multiple components of the Type I CRISPR-Cas complexes, as desired.

Conventional viral and non-viral based gene transfer methods can be used to introduce nucleic acids encoding engineered Type I CRISPR-Cas complexes into cells (e.g., animal, plant, fungal, or algal cells) and target tissues and to co-introduce additional nucleotide sequences as desired. Such methods can also be used to administer nucleic acids (e.g., encoding CRISPR-Cas complexes or components thereof) to cells in vitro. In certain embodiments, nucleic acids are administered for in vivo or ex vivo gene therapy uses. Non-viral vector delivery systems include DNA plasmids, naked nucleic acid, and nucleic acid complexed with a delivery vehicle such as a liposome or polymer. Viral vector delivery systems include DNA and RNA viruses, which have either episomal or integrated genomes after delivery to the cell. For a review of gene therapy procedures, see Anderson, Science 256:808-813, 1992; Nabel & Feigner, TIBTECH 11:211-217, 1993; Mitani & Caskey, TIBTECH 11:162-166, 1993; Dillon, TIBTECH 11:167-175, 1993; Miller, Nature 357:455-460, 1992; Van Brunt, Biotechnology 6(10):1149-1154, 1988; Vigne, Restorative Neurology and Neuroscience 8:35-36, 1995; Kremer & Perricaudet, British Medical Bulletin 51(1):31-44, 1995; Haddada et al., in Current Topics in Microbiology and Immunology Doerfler and Bohm (eds.) (1995); and Yu et al., Gene Therapy 1:13-26, 1994.

### Viral Vectors

In some embodiments, the viral vector is selected from an adeno-associated virus (AAV), adenovirus, retrovirus, and lentivirus vector. While the viral vector may deliver any component of the system described herein so long as it provides the desired profile for tissue presence or expression, in some embodiments the viral vector provides for expression of the guide sequence and optionally delivers a repair template. In some embodiments, the viral delivery system is adeno-associated virus (AAV) 2/8. However, in various embodiments other AAV serotypes are used, such as AAV1, AAV2, AAV4, AAVS, AAV6, and AAV8. In some embodiments, AAV6 is used when targeting airway epithelial cells, AAV7 is used when targeting skeletal muscle cells (similarly for AAV1 and AAVS), and AAV8 is used for hepatocytes. In some embodiments, AAV1 and AAVS can be used for delivery to vascular endothelial cells. Further, most AAV serotypes show neuronal tropism, while AAVS also transduces astrocytes. In some embodiments, hybrid AAV vectors are employed. In some embodiments, each serotype is administered only once to avoid immunogenicity. Thus, subsequent administrations employ different AAV serotypes. Additional viral vectors that can be employed are as described in U.S. Pat. No. 8,697,359, which is hereby incorporated by reference in its entirety.

### Non-Viral Vectors

In some embodiments, the delivery system comprises a non-viral delivery vehicle. In some aspects, the non-viral delivery vehicle is lipid-based. In other aspects, the non-viral delivery vehicle is a polymer. In some embodiments, the non-viral delivery vehicle is biodegradable. In embodiments, the non-viral delivery vehicle is a lipid encapsulation system and/or polymeric particle.

Methods of non-viral delivery of nucleic acids include electroporation, nucleofection, lipofection, microinjection, biolistics, virosomes, liposomes, immunoliposomes, polycation or lipid:nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids. In one embodiment, one or more nucleic acids are delivered as mRNA. Use of capped mRNAs to increase translational efficiency and/or mRNA stability is included in some embodiments. In particular examples, ARCA (anti-reverse cap analog) caps or variants thereof are used. See U.S. Pat. Nos. 7,074,596 and 8,153,773, incorporated by reference herein.

Additional exemplary nucleic acid delivery systems include those provided by Lonza (Cologne, Germany), Amaxa Biosystems (Cologne, Germany), Maxcyte, Inc. (Rockville, Md.), BTX Molecular Delivery Systems (Holliston, Mass.) and Copernicus Therapeutics, Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., TRANSFECTAM™, LIPOFECTIN™, and LIPOFECTAMINE™ RNAiMAX). Cationic and neutral lipids that are suitable for efficient receptor-recognition lipofection of polynucleotides include those of Feigner, WO 91/17424 and WO 91/16024. Delivery can be to cells (ex vivo administration) or target tissues (in vivo administration).

### Lipid-Based and Polymeric Non-Viral Vectors

In certain embodiments, the delivery system comprises lipid particles as described in Kanasty (Nat Mater. 12(11):967-77, 2013), which is hereby incorporated by reference. In some embodiments, the lipid-based vector is a lipid nanoparticle, which is a lipid particle between about 1 and about 100 nanometers in size.

In some embodiments, the lipid-based vector is a lipid or liposome. Liposomes are artificial spherical vesicles comprising a lipid bilayer.

In some embodiments, the lipid-based vector is a small nucleic acid-lipid particle (SNALP). SNALPs comprise small (less than 200 nm in diameter) lipid-based nanoparticles that encapsulate a nucleic acid. In some embodiments, the SNALP is useful for delivery of an RNA molecule such as crRNA. In some embodiments, SNALP formulations deliver nucleic acids to a particular tissue in a subject, such as the liver.

In some embodiments, the guide sequence and/or nucleic acid editing system (or the RNA encoding the same) is delivered via polymeric vectors. In some embodiments, the polymeric vector is a polymer or polymerosome. Polymers encompass any long repeating chain of monomers and include, for example, linear polymers, branched polymers, dendrimers, and polysaccharides. Linear polymers comprise a single line of monomers, whereas branched polymers include side chains of monomers. Dendrimers are also branched molecules, which are arranged symmetrically around the core of the molecule. Polysaccharides are polymeric carbohydrate molecules, and are made up of long monosaccharide units linked together. Polymersomes are artificial vesicles made up of synthetic amphiphilic copolymers that form a vesicle membrane, and may have a hollow or aqueous core within the vesicle membrane.

Various polymer-based systems can be adapted as a vehicle for administering RNA encoding the nucleic acid editing machinery. Exemplary polymeric materials include poly(D,L-lactic acid-co-glycolic acid) (PLGA), poly(caprolactone) (PCL), ethylene vinyl acetate polymer (EVA), poly(lactic acid) (PLA), poly(L-lactic acid) (PLLA), poly(glycolic acid) (PGA), poly(L-lactic acid-co-glycolic acid) (PLLGA), poly(D,L-lactide) (PDLA), poly(L-lactide) (PLLA), PLGA-b-poly(ethylene glycol)-PLGA (PLGA-bPEG-PLGA), PLLA-bPEG-PLLA, PLGA-PEG-maleimide (PLGA-PEG-mal), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (polyacrylic acids), and copolymers and mixtures thereof, polydioxanone and its copolymers, polyhydroxyalkanoates, polypropylene fumarate), polyoxymethylene, poloxamers, poly(ortho)esters, poly(butyric acid), poly(valeric acid), poly(lactide-co-caprolactone), trimethylene carbonate, polyvinylpyrrolidone, polyorthoesters, polyphosphazenes, Poly([beta]-amino esters (PBAE), and polyphosphoesters, and blends and/or block copolymers of two or more such polymers. Polymer-based systems may also include Cyclodextrin polymer (CDP)-based nanoparticles such as, for example, CDP-admantane (AD)-PEG conjugates and CDP-AD-PEG-transferrin conjugates.

Exemplary polymeric particle systems for delivery of drugs, including nucleic acids, include those described in U.S. Pat. Nos. 5,543,158, 6,007,845, 6,254,890, 6,998,115, 7,727,969, 7,427,394, 8,323,698, 8,071,082, 8,105,652, US 2008/0268063, US 2009/0298710, US 2010/0303723, US 2011/0027172, US 2011/0065807, US 2012/0156135, US 2014/0093575, WO 2013/090861, each of which are hereby incorporated by reference in its entirety.

In one embodiment, the delivery system is a layer-by-layer particle system comprising two or more layers. In a further embodiment, the guide RNA and the nucleic acid editing system are present in different layers within the layer-by-layer particle. In a yet further embodiment, the guide RNA and nucleic acid editing system may be administered to a subject in a layer-by-layer particle system such that the release of the guide RNA and nucleic acid editing system from the particles can be controlled in a cell-specific and/or temporal fashion. In one embodiment, the layer-by-layer particle system is designed to allow temporally controlled expression of the guide RNA and the nucleic acid editing system as disclosed herein. Layer-by-layer particle systems are disclosed, for example, in US 2014/0093575, incorporated herein by reference in its entirety.

### Lipid Encapsulation System Vectors

In some embodiments, the lipid-based delivery system comprises a lipid encapsulation system. The lipid encapsulation system can be designed to drive the desired tissue distribution and cellular entry properties, as well as to provide the requisite circulation time and biodegrading character. The lipid encapsulation may involve reverse micelles and/or further comprise polymeric matrices, for example as described in U.S. Pat. No. 8,193,334, which is hereby incorporated by reference. In some embodiments, the particle includes a lipophilic delivery compound to enhance delivery of the particle to tissues, including in a preferential manner. Such compounds are disclosed in US 2013/0158021, which is hereby incorporated by reference in its entirety. Such compounds may generally include lipophilic groups and conjugated amino acids or peptides, including linear or cyclic peptides, and including isomers thereof. An exemplary compound is referred to as cKK-E12, which can affect delivery to liver and kidney cells, for example. The present disclosure can employ compounds of formulas (I), (II), (III), (IV), (V), and (VI) of US 2013/0158021. Compounds can be engineered for targeting to various tissues, including but not limited to pancreas, spleen, liver, fat, kidneys, uterus/ovaries, muscle, heart, lungs, endothelial tissue, and thymus.

In some embodiments, the lipid encapsulation comprises one or more of a phospholipid, cholesterol, polyethylene glycol (PEG)-lipid, and a lipophilic compound. In some embodiments, the lipophilic compound is C12-200, particularly in embodiments that target the liver (Love et al., PNAS 107(5):1864-1869; 2010 (erratum in PNAS 107(21), 2010), incorporated herein by reference in its entirety). In other embodiments, the lipophilic compound C12-200 is useful in embodiments that target fat tissue. In still other embodiments, the lipopeptide is cKK-E12 (Dong et al., PNAS 111(11):3955-3960, 2014, incorporated herein by reference in its entirety).

In some embodiments, the lipid encapsulation comprises 1,2-dioleoyl-sn-glycero-3-phosphoethanolamine (DOPE), cholesterol, C14-PEG2000, and cKK-E12, which as disclosed herein, provides for efficient in vivo editing in liver tissue.

### Additional Components and Features of Non-viral Vectors

When used, delivery particles, whether lipid or polymeric or both, may include additional components useful for enhancing the properties for in vivo nucleic acid delivery (including compounds disclosed in U.S. Pat. No. 8,450,298 and US 2012/0251560, which are each hereby incorporated by reference).

The delivery vehicle may accumulate preferentially in certain tissues thereby providing a tissue targeting effect, but in some embodiments, the delivery vehicle further comprises at least one cell-targeting or tissue-targeting ligand. Functionalized particles, including exemplary targeting ligands, are disclosed in US 2010/0303723 and 2012/0156135, which are hereby incorporated by reference in their entireties.

A delivery vehicle can be designed to drive the desired tissue distribution and cellular entry properties of the delivery systems disclosed herein, as well as to provide the requisite circulation time and biodegrading character. For example, lipid particles can employ amino lipids as disclosed US 2011/0009641, which is hereby incorporated by reference.

The lipid or polymeric particles may have a size (e.g., an average size) in the range of about 50 nm to about 5 μm. In some embodiments, the particles are in the range of about 10 nm to about 100 μm, or about 20 nm to about 50 μm, or about 60 nm to about 5 μm, or about 70 nm to about 500 nm, or about 70 nm to about 200 nm, or about 50 nm to about 100 nm. Particles may be selected so as to avoid rapid clearance by the immune system. Particles may be spherical, or non-spherical in certain embodiments.

In some embodiments, the non-viral delivery vehicle may be a peptide, such as cell-penetrating peptides or cellular internalization sequences. Cell-penetrating peptides are small peptides that are capable of translocating across plasma membranes. Exemplary cell-penetrating peptides include, but are not limited to, Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, I-IN-1, BGSC (Bis-Guanidinium-Spermidine-Cholesterol, and BGTC (Bis-Guanidinium-Tren-Cholesterol).

### VI. Administration

The delivery vehicles (whether comprising conjugates, viral or non-viral vectors, or a combination thereof) may be administered by any method known in the art, including injection, optionally by direct injection to target tissues, specific target cells, and even to specific organelles within a single cell (e.g., the nucleus). In some embodiments, the guide sequence, nucleic acid editing system, and, optionally, repair template are administered simultaneously in the same or in different delivery vehicles. In other embodiments, the guide sequence and nucleic acid editing system and, optionally, repair template are administered sequentially via separate delivery vehicles. In some embodiments, the guide sequence is administered 1-30 days (for example, 1, 3, 5, 7, 10, 14, or 30 days) prior to administration of the nucleic acid editing system, such that the guide sequence accumulates in the target tissue prior to administration of the nucleic acid editing system. In some embodiments, the guide sequence and/or nucleic acid editing system is administered in a plurality of doses, such as 2, 3, 4, 5, 6, 7, 8, 9, 10, 11, 12, 13, 14, 15, 16, 17, 18, 19, 20, or more doses. In various embodiments, the guide sequence and/or nucleic acid editing system is administered over a time period of from one week to about six months, such as from about two to about ten doses within about two months, such as from about three to about five doses over about one month.

In one embodiment, the guide sequence and, optionally, a repair template, are provided in an AAV vector that is administered to the subject or cell prior to administration of a nanoparticle containing the nucleic acid editing system. In a further embodiment, the AAV vector comprising the guide sequence is administered 3, 4, 5, 6, 7, 8, 9, or 10 days prior to the administration of the nanoparticle, to allow expression of the guide sequence from the AAV vector. In a yet further embodiment, the nanoparticle containing the nucleic acid editing system is administered multiple times, for example, once every 1, 2, 3, 4, 5, 6, 7, 8, 9, 10, 11, 12, 13, 14, or 15 days. In a still further embodiment, the nanoparticle containing the guide sequence is administered for 1 month, 2 months, 3, months, 4 months, 6 months, 8 months, 10 months, 12 months, 18 months, 24 months, or longer. Since AAV expression can occur for 2 years or longer, in one embodiment, the expression of the guide sequence and, optionally, repair template, from the AAV vector and the continual administration of nanoparticles containing the nucleic acid editing system provides efficient gene editing of the target sequence with reduced or absent off-target effects due to the transient expression of the nucleic acid editing system.

In another embodiment, the repair template is delivered via an AAV vector, and is injected 3, 4, 5, 6, 7, 8, 9, or 10 days prior to the administration of nanoparticles containing the nucleic acid editing system and/or the guide sequence. As described above, the nanoparticles may be administered multiple times, and for several months. In such embodiments, the repair template is expressed from the AAV vector in the cell for 2 years or longer, and the nanoparticles comprising the nucleic acid editing system and/or guide sequence are administered in multiple administrations over time in order to provide efficient gene editing of the target sequence with reduced or absent off-target effects.

In particular embodiments, one or more guide sequences and, optionally, a repair template, is provided in an AAV vector that is administered first, and a bi-directional Type I CRISPR nucleic acid editing system in a lipid-based delivery vehicle is subsequently administered in one or more doses. In some embodiments, the bi-directional Type I CRISPR nucleic acid editing system is administered in a lipid-based delivery vehicle about 7 days and about 14 days after the administration of the one or more guide sequences in an AAV vector.

In another embodiment, each of the components of the delivery systems provided herein (e.g., the nucleic acid editing system, guide sequence and, optionally, repair template) are each contained in the same or in different nanoparticles. In a further embodiment, the nanoparticles containing the nucleic acid editing system, guide sequence, and, optionally, repair template, are administered at multiple time points, for example, every 2, 3, 4, 5, 6, 7, 8, 9, 10, 11, or 12 days. In another embodiment, the administration of the nanoparticles separately comprising the nucleic acid editing system and guide sequence are administered at different time points in order to enhance gene editing efficiency in a particular cell or for a particular disease type.

In some embodiments, the administration of the delivery system is controlled so that expression of the nucleic acid editing system is transient. In some embodiments, such transient expression of the nucleic acid editing system minimizes off-target effects, thereby increasing the safety and efficiency of the gene editing system disclosed herein. For example, expression of the nucleic acid system is controlled via selection of the delivery vehicles and/or promoters disclosed herein or known to those of skill in the art.

In another embodiment, the guide sequence, bi-directional Type I CRISPR nucleic acid editing complexes, Cas3 nuclease, and, optionally, repair template, are administered to a subject or a cell at the same time, such as on the same delivery vehicle, and one or more component (for instance, the guide sequence, Cas3 nuclease, CRISPR proteins, and/or repair template) is under the control of an inducible promoter. As an example, in one embodiment, the guide sequence, bi-directional Type I CRISPR nucleic acid editing complexes, and repair template are each present on an AAV viral vector, and the guide sequence is under the control of an inducible promoter, for example, a small molecule-induced promoter such as tetracycline-inducible promoter. In a further embodiment, components of the bi-directional Type I CRISPR nucleic acid editing system are expressed 5-7 days following administration of the vector, after which the expression of the guide sequence is induced by one or more injections of the small molecule such as tetracycline. The guide sequence expression can be induced at various time points in order to increase gene editing efficiency; for example guide sequence expression may be induced every day, or every 2 days, or every 3 days, or every 5 days, or every 10 days, or every 2 weeks, for at least 1 week or at least 2 weeks, or at least 3 weeks, or at least 4 weeks, or at least 5 weeks, or at least 6 weeks, or at least 7 weeks, or at least 8 weeks, or at least 10 weeks, or at least 11 weeks, or at least 12 weeks, or more. Thus, bi-directional Type I CRISPR nucleic acid editing system component(s) may be expressed from the AAV vector over time, and the guide sequence may be inducibly expressed by multiple injections of the inducing molecule over several days, weeks, or months. Similarly, the guide sequence can be expressed from the AAV vector over time, and components of the bi-directional Type I CRISPR complexes may be inducibly expressed under control of an inducible promoter by multiple injections of the inducing molecule over several days, weeks, or months.

In another embodiment, one or more guide sequences and, optionally, a repair template, is delivered via an RNA conjugate, such as an RNA-GalNAc conjugate, and the nucleic acid editing system is delivered via a viral or non-viral vector, such as a nanoparticle. In another embodiment, the guide sequence and repair template are attached to the nanoparticle comprising the nucleic acid editing system, such that the components are delivered to the target cell or tissue together. In such embodiments, the guide sequence, repair template, and nucleic acid editing system may be delivered to the target cell or tissue together, and expression of each component may be controlled by way of different promoters, including inducible promoters, as disclosed herein.

In one aspect, the present disclosure provides methods for modifying a target polynucleotide in a cell, which may be in vivo, ex vivo, or in vitro. In some embodiments, the one or more delivery vehicles comprising a nucleic acid editing system and/or guide sequence and, optionally, repair template, are administered to a subject. In further embodiments, the nucleic acid editing system and guide sequence and, optionally, repair template, are targeted to one or more target tissues in the subject. For example, in one embodiment, the target tissue is liver, endothelial tissue, lung (including lung epithelium), kidney, fat, or muscle. In one embodiment, the one or more delivery vehicles comprise a viral vector (e.g., AAV) or a non-viral vector such as, for example, MD-1, 7C1, PBAE, C12-200, cKK-E12, or a conjugate such as a cholesterol conjugate or an RNA conjugate as disclosed herein. In one embodiment, the target tissue is liver, and one or more delivery vehicle is MD-1. In another embodiment, the target tissue is endothelial tissue, and one or more delivery vehicle is 7C1. In another embodiment, the targeting tissue is lung, and one or more delivery vehicle is PBAE or 7C1. In another embodiment, the target tissue is kidney, one or more delivery vehicle is an RNA conjugate. In another embodiment, the target tissue is fat, and one or more delivery vehicle is C12-200. In another embodiment, the target tissue is muscle (e.g., skeletal muscle) and one or more delivery vehicle is a cholesterol conjugate.

The delivery vehicles (whether viral vector or non-viral vector or RNA conjugate material) may be administered by any method known in the art, including injection, optionally by direct injection to target tissues. Nucleic acid modification can be monitored over time by, for example, periodic biopsy with PCR amplification and/or sequencing of the target region from genomic DNA, or by RT-PCR and/or sequencing of the expressed transcripts. Alternatively, nucleic acid modification can be monitored by detection of a reporter gene or reporter sequence. Alternatively, nucleic acid modification can be monitored by expression or activity of a corrected gene product or a therapeutic effect in the subject.

In some embodiments, the subject is a human in need of therapeutic or prophylactic intervention. Alternatively, the subject is an animal, including livestock, poultry, domesticated animal, or laboratory animal. In various embodiments, the subject is a mammal, such as a human, horse, cow, dog, cat, rodent, or pig. Also contemplated are embodiments where the “subject” is a fungus or a plant, and the bi-directional Type I CRISPR nucleic acid editing systems described herein are used to edit the genome of these organisms.

In some embodiments, the methods provided herein include obtaining a cell or population of cells from a subject and modifying a target polynucleotide in the cell or cells ex vivo, using the editing systems, compositions, methods, and/or kits disclosed herein. In further embodiments, the ex vivo-modified cell or cells may be re-introduced into the subject following ex vivo modification. Thus, the present disclosure provides methods for treating a disease or disorder in a subject, comprising obtaining one or more cells from the subject, modifying one or more target nucleotide sequences in the cell ex vivo, and re-introducing of the cell with the modified target nucleotide sequence back into the subject having the disease or disorder. In some embodiments, cells in which nucleotide sequence modification has occurred are expanded in vitro prior to reintroduction into the subject having the disease or disorder. In one embodiment, the cells are bone marrow cells.

In other embodiments, the nucleic acid editing system and guide sequence and, optionally, repair template, are administered to a cell in vitro.

In some embodiments, at least one component of the delivery system (e.g., the guide sequence or the nucleic acid editing system) accumulates in the target tissue, which may be, for example, liver, heart, lung (including airway epithelial cells), skeletal muscle, CNS (e.g., nerve cells), endothelial cells, blood cells, bone marrow cells, blood cell precursor cells, stem cells, fat cells, or immune cells. Tissue targeting or distribution can be controlled by selection and design of the viral vector, or in some embodiments is achieved by selection and design of lipid or polymeric particles. In some embodiments, the desired tissue targeting of the activity is provided by the combination of viral and non-viral delivery vehicles.

### Direct Protein or Nucleoprotein Complex Delivery

Also contemplated are methods of directly delivering fully assembled nucleoprotein complex(es), or subunits thereof, to a target cell or organelle within the cell (e.g., directly to the nucleus of a eukaryotic cell).

By way of example, NLS-tagged CRISPR-Cascade or other Type I complexes designed to target opposite stands of a DNA target are recombinantly expressed and purified. NLS-tagged Cas3 is recombinantly expressed and purified separately, or Cas3 is tethered to one of the Cas proteins in the crRNA-guided surveillance complex. For example, Cas3 is in some embodiments fused to Cse1 in Cascade. In some examples, the protein(s) and RNA (e.g., sgRNA, crRNA) are assembled as a complex in vitro and the purified complex is delivered to a cell.

A pair of NLS-tagged CRISPR-Cascade or other Type I complexes designed to target flanking locations on opposite stands of a DNA target are co-injected or transfected with Cas3 into either the nuclease or the cytoplasm of a eukaryotic cell. The concentration of each protein or complex injected may be adjusted to limit toxicity and off-target effects. Methods of microinjection into individual cells, or into subcellular organelles (such as the nucleus) are well known in the art; see for instance Microinjection, (eds. Lacal, Perona & Feramisco), Birkhäuser Verlag, 1999, and Komarova et al., “Microinjection pf Protein Samples,” Chapter 5 in Live Cell Imaging (eds. Goldman & Spector), CSHL Press, 2005. Microinjection devices are commercially available, for instance from Tritech Research (Los Angeles, Calif.).

### VII. Genome Editing and Modification

The bi-directional Type I CRISPR complex systems disclosed herein can be used for genome editing, gene modification, drug delivery, and all other uses for which Cas9-mediated CRISPR systems have been adopted. See, for instance, U.S. Pat. No. 8,697,359, which is incorporated herein by reference in its entirety

### Repair Templates

In certain instances, particularly where a nucleotide substitution or insertion is desired, for example, the compositions, methods, kits, and delivery systems provided herein further comprise a repair template. Repair templates may comprise any nucleic acid, for example, DNA, messenger RNA (mRNA), small interfering RNA (siRNA), microRNA (miRNA), single stranded RNA (ssRNA), or antisense oligonucleotides. In some embodiments, the repair template is a double stranded DNA repair template. The basic components and structure of the DNA repair template to support gene editing is known, and described in Ran (PNAS 8(11):2281-2308, 2013); and Pyzocha et al. (Methods Mol. Biol. 1114:269-277, 2014) which are hereby incorporated by reference.

The length of the repair template can vary, and can be, for example, from 200 base pairs (bp) to about 5000 bp, such as about 200 bp to about 2000 bp, such as about 500 bp to about 1500 bp. In some embodiments, the length of the DNA repair template is about 200 bp, or is about 500 bp, or is about 800 bp, or is about 1000 bp, or is about 1500 bp. In other embodiments, the length of the repair template is at least 200 bp, or is at least 500 bp, or is at least 800 bp, or is at least 1000 bp, or is at least 1500 bp in length.

In some embodiments, the repair template is provided in the same delivery vehicle as the guide sequence(s). In other embodiments, the repair template is in the same delivery vehicle as the nucleic acid editing system (that is, the bi-directional Type I CRISPR-Cas system). In some embodiments, the repair template can be present on a contiguous polynucleotide with the guide sequence, and the repair template may be designed for incorporation by homologous recombination.

In some embodiments, the delivery system provides a guide sequence and repair template, wherein the repair template is covalently or non-covalently bound to the guide sequence. In further embodiments, the repair template is partially annealed to the guide sequence. In some embodiments, the repair template is covalently or non-covalently bound to a guide sequence delivered via a viral vector and the nucleic acid editing system comprises a nuclear localization signal and is delivered by a non-viral vector, such that it carries the guide sequence along with the repair template to the nucleus of the cell. Thus, in some embodiments, the delivery systems, compositions, methods, and kits disclosed herein greatly improve the efficiency of nucleotide sequence modification by providing a system by which the repair template is efficiently directed to the nucleus of the cell.

### Diseases and Disorders

While the bi-directional Type I CRISPR-Cas nucleic acid modification/editing systems described herein can be used to make essentially any desired change, in some embodiments the subject has a genetic disorder which is sought to be corrected. In some aspects, the disorder is an inborn error of metabolism. In other embodiments, the nucleic acid modification provides a loss of function for a gene that is deleterious. In some embodiments, the inborn error of metabolism can be selected from disorders of amino acid transport and metabolism, lipid or fatty acid transport and metabolism, carbohydrate transport and metabolism, and metal transport and metabolism. In some embodiments, the disorder is cancer-related. In some embodiments, the disorder is hemophilia, cystic fibrosis, or sickle cell disease. Exemplary diseases and conditions that may be treated, prevented or alleviated with the delivery systems, compositions, kits, and methods provided herein include: cystic fibrosis, hemophilia, Huntington Disease, de Grouchy Syndrome, Lesch-Nyhan Syndrome, galactosemia, Gaucher Disease, CADASIL Disease, Tay-Sachs Disease, Fabry Disease, color blindness, cri du chat, Duchenne muscular dystrophy, 22q11.2 deletion syndrome, Angelman syndrome, Canavan disease, Charcot-Marie-Tooth disease, Down syndrome, Klinefelter syndrome, neurofibromatosis, Prader-Willi syndrome, Tay-Sachs disease, haemochromatosis, phenylketonuria, polycystic kidney disease, sickle cell disease, progeria, alpha 1-antitrypsin deficiency (A1AD), and tyrosinemia, growth hormone deficiency, metachromatic leukodystrophy, mucopolysaccharidosis I, phenylketonuria, short chain acyl-CoA dehydrogenase deficiency, alpha-1 antitrypsin deficiency, diabetes, obesity, myocarditis, glomerulonephritis, organophosphate toxicity, xenotransplantation, hypoxic-ischemia encephalopathy, liver regeneration, and various types of cancer, among others. Exemplary genetic disorders that can be treated or ameliorated in various embodiments, as well as target genes that can be edited for improved or reduced activity, are disclosed in U.S. Pat. No. 8,697,359, which are hereby incorporated by reference.

In one aspect, the delivery systems, compositions, methods, and kits disclosed herein are useful for therapeutic treatment of genetic diseases and disorders, cancers, immune system disorders, or infectious diseases. In some embodiments, the diseases, disorders, and cancers are associated with mutations that cause expression of one or more defective gene products, or cause an aberrant increase or decrease in the production of a gene product. In some embodiments, the therapeutic efficacy of the delivery systems, compositions, methods, and kits disclosed herein may be assessed or measured by expression level or activity level of the product of the targeted nucleotide sequence. In some embodiments, gene loci are sequenced by Sanger or Next Generation

Sequencing. In some embodiments, in human subjects or other subjects, a therapeutic effect or the therapeutic efficiency of the compositions and methods for target sequence modification disclosed herein may be measured or monitored using surrogate markers of efficiency. Surrogate markers of efficiency may be, for example, an improvement in a symptom of the disease or condition; a clinical marker such as, for example, liver function; expression of a wild-type gene product or an improved gene product relative to the gene product that was expressed in the cell or subject prior to treatment; expression of a sufficient amount or activity of the gene product to improve or resolve the disease or disorder; or expression of the gene product in a manner that provides any other therapeutic effect. In some embodiments, surrogate markers may include serum markers such as, for example, factor VIII and/or IX. For example, factor VIII and factor IX can be measured as surrogate markers for efficiency of treatment for hemophilia A and hemophilia B, respectively. In some embodiments, any gene product excreted through exosome to the serum can be detected by purification and sequencing.

In some embodiments, the disease or disorder may be therapeutically treated using the methods, compositions, kits, and delivery systems disclosed herein, wherein an efficiency rate of target sequence modification or an efficiency rate of gene product modification is less than 100%, or wherein an effect on fewer than 100% of the cells in the relevant tissue, has a therapeutic effect in the subject. For example, a therapeutic effect may be achieved when the percent efficiency of nucleic acid modification may be about 0.01% to about 50%, about 0.05% to about 40%, about 0.1% to about 30%, about 0.5% to about 25%, about 1% to about 20%, about 1% to about 15%, about 1% to about 10%, or about 1% to about 5%. Thus, even if the efficiency of nucleotide sequence modification is relatively low (e.g., less than 50%, or less than 40%, or less than 30%, or less than 20%, or less than 10%, or less than 5%, or less than 1%, or less than 0.5%, or less than 0.1%), modest expression of the introduced or corrected or modified gene product may result in a therapeutic effect in the disease or disorder.

Thus, in some embodiments, a genetic disease or condition may be improved or resolved even if the target nucleotide sequence is only modified in a fraction of the target population of cells in the subject. In some embodiments a percent efficiency of nucleic acid modification of less than 50%, less than 40%, less than 30%, less than 20%, less than 10%, less than 5%, less than 1%, less than 0.5%, or less than 0.1% nevertheless results in high level expression of an introduced or corrected gene and thereby resolves the genetic disorder by providing sufficient expression of the relevant product.

In some genetic disorders, the presence of only a few improved or corrected gene products results in a measurable improvement or even resolution of the disease or disorder. For example, without wishing to be bound by theory, disorders in which disease is caused by a simple deficiency of a particular gene product may be resolved with a limited number of nucleotide sequence modifications. For example, recessively inherited disorders are often simple loss-of-function mutations, and often there is a wide variation in the normal levels of gene expression (e.g., heterozygotes often have about 50% of the normal gene product and are asymptomatic), such that expression of a relatively small percentage of the normal gene product may be sufficient to resolve the disorder. On the other hand, dominantly inherited disorders in which heterozygotes exhibit loss-of-function with 50% of the normal gene product may, in some embodiments, require a higher level of nucleotide sequence modification in order to achieve a therapeutic effect. For example, and without wishing to be bound by theory, disorders such as cystic fibrosis and Muscular dystrophy (MD) may exhibit a therapeutic effect upon an efficiency of about 1% to about 40%; hemophilia A and B, galactosemia, primary hyperoxaluria, hepatoerythropoietic porphyria, and Wilson's disease may each exhibit a therapeutic effect upon achieving an efficiency of about 1% to about 5%; and alpha 1-antitrypsin deficiency, hereditary tyrosinemia type I, Fanconi's anemia, and junctional epidermolysis bullosa may each exhibit a therapeutic effect upon achieving an efficiency of about 0.1% to about 5%. A percent efficiency of nucleic acid modification may be directly measured in animal models or in in vitro assays by measuring the percent of cells in the target population in which the target nucleotide sequence has been modified. Or, a percent efficiency of nucleic acid modification may be indirectly measured, such as by using surrogate markers as described above.

In some embodiments, the method modifies a target sequence that is a genetic variant selected from a single-nucleotide polymorphism (SNP), substitution, insertion, deletion, transition, inversion, translocation, nonsense, missense, and frame shift mutation. In other embodiments, the target sequence is a sequence from an infectious agent, such as a virus or provirus. A provirus is a viral genome that has integrated into the DNA of a host cell. Proviruses may be retroviruses or other types of viruses that are capable of integration into a host genome. For example, adeno-associated viruses (AAV) have been shown to be capable of host chromosome integration. Other proviruses include, without limitation, HIV and HTLV.

In some embodiments, the delivery systems and compositions disclosed herein are formulated such that the ratio of the components is optimized for consistent delivery to the target sequence and/or consistent resolution of the disease or disorder. In one embodiment, the ratio of the crRNA and nucleic acid editing system is optimized for consistent delivery to the target sequence and/or consistent resolution of the disease or disorder. In another embodiment, the ratio of the repair template to the guide sequence and/or to the nucleic acid editing system is optimized for consistent delivery to the target sequence and/or consistent resolution of the disease or disorder. For example, in some embodiments, the delivery systems provide expression of an optimal number of guide sequences such that upon delivery to the cell, target tissue, or subject, the modification of target nucleotide sequences by the guide sequence and nucleic acid editing system and, optionally, repair sequence, can be maximized

In one aspect, the present disclosure provides methods for safe and efficient delivery of a nucleic acid editing system via a non-viral vector delivery system, or via a system that includes a viral vector as well as a non-viral vector, such that off-target effects (e.g., off-target effects due to long term expression of a nucleic acid editing system and a guide sequence through genome integration of an AAV vector delivery system) are minimized

### VIII. Kits

In one aspect, the disclosure provides kits containing any one or more of the components disclosed in the above methods, compositions, and gene editing systems. Kit components may be provided individually or in combinations, and may be provided in any suitable container, such as a vial, a bottle, or a tube. In some embodiments, the kits disclosed herein comprise one or more reagents for use in the embodiments disclosed herein. For example, a kit may provide one or more reaction or storage buffers. Reagents may be provided in a form that is usable in a particular assay, or in a form that requires addition of one or more other components before use (e.g., in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof. In some embodiments, the buffer is alkaline. In some embodiments, the buffer has a pH from about 7 to about 10.

For example, a kit may comprise: (1) a first and second guide sequence and (2) a nucleic acid editing system. The kit may further comprise a repair template. The kit may provide (1) an expression system providing for expression of a guide sequence in a target cell or target tissue for at least 2 weeks, the guide sequence directing cleavage of a target nucleic acid sequence in the target tissue by a nucleic acid editing system, and the expression system optionally comprising a repair template, and (2) one or more doses of an RNA delivery system, each dose providing for expression of the nucleic acid editing system in the target tissue for no more than about one month. In various embodiments, the kit may provide from two to ten doses of the gene editing system or components thereof, which may be administered over a time period of from one week to about two months. In some embodiments, the kit contains from about two to about five unit doses.

The kit may be custom made to repair a genetic disorder, such as is an inborn error of metabolism. In other embodiments, the nucleic acid modification provides a loss of function for a gene that is deleterious. In some embodiments, the inborn error of metabolism can be selected from disorders of amino acid transport and metabolism, lipid or fatty acid transport and metabolism, carbohydrate transport and metabolism, and metal transport and metabolism. In some embodiments, the disorder is hemophilia, cystic fibrosis, or sickle cell disease.

### IX. Engineered Type I CRISPR-Cas Complexes

Optionally, the systems described herein can employ engineered, non-naturally occurring Type I CRISPR-Cas complex(es), for instance one that is simplified in its genetic footprint and yet can recognize one or more complementary nucleic acid sequences with a high degree of specificity, such as a partially or fully concatenated CRISPR-Cas complex.

In some embodiments, the engineered complex includes one or more subunits Cse1, Cse2, Cas7, Cas5, and Cas6 (for a Cascade complex), or one or more subunits Csy1, Csy2, Csy3, and Csy4 (for a Csy complex). Optionally, the Cas3 nuclease may be included in the engineered complex. In some embodiments, the engineered complexes includes crRNA. In some embodiments, the engineered complex, or one or more subunits within the complex, is modified to contain a non-native, non-naturally occurring NLS. In some embodiments, two or more subunits of the complex are linked to each other.

Also contemplated are engineered, non-naturally occurring Type I CRISPR-Cas complexes wherein the nucleotide sequence in the crRNA is modified. In some embodiments, the engineered complex contains additional nucleotides in the crRNA. For instance, in some embodiments, additional Cse2 subunits are added to a CRISPR-Cascade complex containing the extended crRNA complex. In some embodiments, the engineered complex contains a NLS.

### Reduced Number of Open Reading Frames

The proteins that make up Cascade (Cse1, Cse2, Cas7, Cas5e, and Cas6e; some of which have multiple units) or the proteins that make up Csy (Csy1, Csy2, Csy3, and Csy4; some of which have multiple units) can be separately encoded for, with stop codons indicating the terminal end of each protein. Accordingly, to express the native structure, there are at least five open reading frames corresponding to each protein. Referring to FIG. 1A, the native Type IE CRISPR-Cascade operon from E. coli containing five open reading frames is shown, with a separate open reading frame corresponding to each protein and where each protein is color coded. Native Type IF CRISPR-Csy is similarly illustrated in FIG. 3.

Through linkage of CRISPR associated (Cas) genes, the number of open reading frames (ORFs) required to express Cascade or Csy (that is, a Type I CRISPR-Cas complex) is reduced. Reducing the number of open reading frames reduces the genetic footprint, thereby simplifying expression. In some embodiments, reduction of ORFs is achieved through linkage of two or more subunits. Subunit linkage allows for transcription and expression of the two or more linked proteins in the same open reading frame. In some embodiments, the number of ORFs is reduced from 5 to 4, from 5 to 3, from 5 to 2, or from 5 to 1. In a preferred embodiment, the number of ORFs is reduced from 5 to 3. A linker is any amino acid sequence that connects subunits that were previous encoded on separate open reading frames.

### Linkage of Two or More Type I CRISPR-Cas Subunits

Multi-subunit crRNA-guided Type I complexes (e.g. Cascade and Csy) use sophisticated mechanisms for recognition of and assembly around invading DNA. Cascade is composed of 11 subunits, which assemble around a nucleic acid target (FIG. 1A). The 11 protein subunits of Cascade include Cse1 (1 subunit), Cse2 (2 subunits), Cas7 (6 subunits), Cas5e (1 subunit), and Cas6e (1 subunit).

TABLE 1
Cascade proteins, subunits, and assigned numerical value.

| | | Numerical |
|---|---|---|
| Protein Subunit | | Assignment |
| Cse1 | | 1 |
| Cse2 | Cse2.1 | 2 |
| | Cse2.2 | 3 |
| Cas7 | Cas7.1 | 4 |
| | Cas7.2 | 5 |
| | Cas7.3 | 6 |
| | Cas7.4 | 7 |
| | Cas7.5 | 8 |
| | Cas7.6 | 9 |
| Cas5e | | 10 |
| Cas6e | | 11 |

For purposes of this section of the disclosure, the 11 protein subunits are numerically labeled 1 through 11 and defined in Table 1 above. In some embodiments, one or more subunits are linked using a linker. In some embodiments, the subunits linked include the following: 1-2; 2-3; 3-4; 4-5; 5-6; 6-7; 7-8; 8-9, 9-10, or 10-11, and/or combinations thereof. In some embodiments, linkage occurs between subunits: 1-2-3; 1-2-3-4; 1-2-3-4-5; 1-2-3-4-5-6; 1-2-3-4-5-6-7; 1-2-3-4-5-6-7-8; 1-2-3-4-5-6-7-8-9; 1-2-3-4-5-6-7-8-9-10; 1-2-3-4-5-6-7-8-9-10-11; and/or combinations thereof. In some embodiments, linkage occurs between subunits: 2-3-4; 2-3-4-5; 2-3-4-5-6; 2-3-4-5-6-7; 2-3-4-5-6-7-8; 2-3-4-5-6-7-8-9; 2-3-4-5-6-7-8-9-10; 2-3-4-5-6-7-8-9-10-11; and/or combinations thereof. In some embodiments, linkage occurs between subunits: 3-4-5; 3-4-5-6; 3-4-5-6-7; 3-4-5-6-7-8; 3-4-5-6-7-8-9; 3-4-5-6-7-8-9-10, 3-4-5-6-7-8-9-10-11; and/or combinations thereof. In some embodiments, linkage occurs between subunits: 4-5-6; 4-5-6-7; 4-5-6-7-8; 4-5-6-7-8-9; 4-5-6-7-8-9-10; 4-5-6-7-8-9-10-11; and/or combinations thereof. In some embodiments, linkage occurs between subunits: 5-6-7; 5-6-7-8; 5-6-7-8-9; 5-6-7-8-9-10; 5-6-7-8-9-10-11; and/or combinations thereof. In some embodiments, linkage occurs between subunits: 6-7-8; 6-7-8-9; 6-7-8-9-10; 6-7-8-9-10-11; and/or combinations thereof. In some embodiments: 7-8-9; 7-8-9-10; 7-8-9-10-11; and/or combinations thereof. In some embodiments, linkage occurs between subunits: 8-9-10; 8-9-10-11; and/or combinations thereof. In some embodiments, linkages occurs between subunits: 9-10-11; and/or combinations thereof. These embodiments and the linkage indicated therein can be combined in a complex containing two or more linked subunits.

Similarly, the 9 protein subunits of the Csy ribonucleoprotein include Csy1 (1 subunit), Csy2 (1 subunits), Csy3 (6 subunits), and Csy4 (1 subunit). For purposes of this section of the disclosure, the 9 protein subunits are numerically labeled 1 through 9 and defined in Table 2 below. In some embodiments, one or more Csy subunits are tethered using a linker. In some embodiments, the tethered subunits include the following: 1-2; 2-3; 3-4; 4-5; 5-6; 6-7; 7-8; 8-9, and/or combinations thereof. In some embodiments, linkage occurs between subunits: 1-2-3; 1-2-3-4; 1-2-3-4-5; 1-2-3-4-5-6; 1-2-3-4-5-6-7; 1-2-3-4-5-6-7-8; 1-2-3-4-5-6-7-8-9; and/or combinations thereof. In some embodiments, linkage occurs between subunits: 2-3-4; 2-3-4-5; 2-3-4-5-6; 2-3-4-5-6-7; 2-3-4-5-6-7-8; 2-3-4-5-6-7-8-9; and/or combinations thereof. In some embodiments, linkage occurs between subunits: 3-4-5; 3-4-5-6; 3-4-5-6-7; 3-4-5-6-7-8; 3-4-5-6-7-8-9; and/or combinations thereof. In some embodiments, linkage occurs between subunits: 4-5-6; 4-5-6-7; 4-5-6-7-8; 4-5-6-7-8-9; and/or combinations thereof. In some embodiments, linkage occurs between subunits: 5-6-7; 5-6-7-8; 5-6-7-8-9; and/or combinations thereof. In some embodiments, linkage occurs between subunits: 6-7-8; 6-7-8-9; and/or combinations thereof. In some embodiments: 7-8-9; and/or combinations thereof.

TABLE 2
Csy proteins, subunits, and assigned numerical value.

| | | Numerical |
|---|---|---|
| Protein | Subunit | Assignment |
| Csy1 | | 1 |
| Csy2 | | 2 |
| Csy3 | Csy3.1 | 3 |
| | Csy3.2 | 4 |
| | Csy3.3 | 5 |
| | Csy3.4 | 6 |
| | Csy3.5 | 7 |
| | Csy3.6 | 8 |
| Csy4 | | 9 |

### Linker

The subunits of a natural Type I CRISPR-Cas complex assemble in vivo around nucleic acid. In some embodiments, two or more Type I CRISPR-Cas subunits are linked to create an engineered, non-naturally occurring complex. In some embodiments, Type I CRISPR-Cas complex subunits are linked such that the subunits are connected. In some embodiments, subunits are linked using a chemical compound. In some embodiments, the linker is an inorganic compound. In some embodiments, the linker is an organic compound. In some embodiments, the linker is a hybrid organic and inorganic compound. In some embodiments, the linker is covalently bonded to two or more subunits.

In some embodiments, two or more subunits are cross-linked. Cross-linking two or more proteins is a technique known to one skilled in the art that has not yet been adapted to CRISPR-Cas systems. To that point, commercially available kits can be purchased, e.g. ThermoScientific™ Peirce® (Controlled Protein-Protein Crosslinking Kit), to tether protein subunits. Chemical crosslinking reagents covalently bind proteins, domains, or peptides possessing reactive functional groups (amines, sulfhydryls, carboxyls, carbonyls, and hydroxyls) and have variable spacer arm length, among other characteristics (available on the World Wide Web at piercenet.com/method/crosslinking-protein-interaction-analysis).

In some embodiments, the linker is translationally fused to two or more subunits. In some embodiments, two or more subunits are genetically fused. In some embodiments, linkage occurs from about the 3′ end of one subunit to about the 5′ prime end of a second subunit. In some embodiments, linkage occurs at a direct path between subunits. In some embodiments, the linker is an amino acid sequence. In some embodiments, amino acids are chosen from one or more of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, and/or combinations thereof. In some embodiments, the linker amino acid sequence is translationally fused to two or more subunits. In some embodiments, the amino acid linker is covalently bonded to two or more subunits.

In some embodiments, two or more subunits are genetically fused using a linker composed of amino acid units. Genetic fusion of two previously unconnected proteins creates an engineered, non-naturally occurring Type I CRISPR-Cas complex wherein the overall genetic footprint is reduced. Genetic fusion of protein subunits of a complex has been performed on other systems and is achievable in the Type I CRISPR-Cas system without undue experimentation by one skilled in the art equipped with knowledge of a preferred linker length based on the x-ray crystal structure. Examples of genetic fusion of proteins using an amino acid sequence include the following, which are herein incorporated by reference in their entirety: (1) Martin, A. et al. Nature 2005 Oct. 20; 437:1115-1120); (2) Wang, F. et al. Nature 2014 Aug. 28; 512:441-444; (3) Schmitz, K. R. and Sauer, R. T. Molecular Microbiology 2014 Jul. 13; 93(4):617-628; (4) Wang, Q. et al., Chem. Commun. 2014 Mar. 3; 50:4299-4301; (5) Andre et al., PNAS 2013 Feb. 19, 110(8):3191-3196; (7) Weidle, U. H. et al., Cancer Genomics and Proteomics 2012 9(6):357-372).

### Nuclear Localization Signal

Viable genome-editing tools must be delivered to the nucleus of eukaryotic cells. A nuclear localization signal or sequence (NLS) is an amino acid sequence that “tags” a protein for import into the cell nucleus by nuclear transport. In some embodiments, this signal consists of one or more short sequences of positively charged lysines or arginines exposed on the protein surface. Clusters of arginines or lysines in nucleus-targeted proteins signal the anchoring of these proteins to specialized transporter molecules found on the complex or in the cytoplasm. In some embodiments, the NLS is genetically linked to one or more of the Cas protein subunits. In some embodiments, the NLS is included in the linker sequence. In some embodiments, the NLS is included as part of the linker sequence that genetically tethers two or more Cas protein subunits.

### Fusion of a Nuclease to a Type I CRISPR-Cas Complex or Polypeptide

A nuclease is an enzyme that can cleave a phosphodiester bond between nucleotide subunits in a sequence of nucleic acids. By cleaving a nucleotide, a nuclease can degrade the activity associated with the sequence. Accordingly, a nuclease can be used for genome editing. In some embodiments, a nuclease is fused to a Type I CRISPR-Cas complex polypeptide. In some embodiments, the fused nuclease can be used for genome editing. In some embodiments, the nuclease is Cas3.

In some embodiments, the nuclease is fused to one or more engineered, non-naturally occurring Cascade or Csy complexes described herein.

Modified crRNA

Type I CRISPR-Cas complex relies on complementary base pairing of at least a portion of 32 nucleotides in the crRNA with target DNA. The nucleotides in the crRNA can be modified to recognize a target nucleic acid sequence. In other systems, the Cas9 guide RNA sequence has been modified to improve nuclease specificity, and this technique can be performed on the crRNA of a Type I CRISPR-Cas complex by one skilled in the art (Fu et al., Nature Biotechnology 32(3):279-284, 2014; herein incorporated by reference in its entirety).

In some embodiments, CRISPR-Cas crRNA is modified. In some embodiments, nucleotides in the crRNA are modified. In some embodiments, crRNA is extended by one or more nucleotides. Extending the number of nucleotides in the Type I CRISPR-Cas surveillance complex that participate in target recognition improves target specificity by increasing the number of nucleotides that recognize complementary nucleic acids. In some embodiments, the number of nucleotides are extended by about 6 nucleotides to about 50 nucleotides. In some embodiments, the nucleotides are extended by about 12 nucleotides. In some embodiments, the nucleotides are extended by about 24 nucleotides. In some embodiments, the extended Cascade includes additional Cse2 subunits. In some embodiments, about one Cse2 subunit is added to an extended Cascade complex. In some embodiments, about two Cse2 subunits are added to an extended Cascade complex.

### Cas Gene Sequences

In some embodiments, the Cas gene sequences are provided for one or more engineered, non-naturally occurring Type I CRISPR-Cas complex described herein. In some embodiments, one or more engineered, non-naturally occurring Cas gene sequences encodes for one or more complexes with two or more linked subunits described herein, for one or more complexes with a modified crRNA described herein, the like, and/or combinations thereof.

### Optimized Codons

A codon for expression of one or more engineered, non-naturally occurring complexes that include clustered regularly interspaced short palindromic repeat-associated complex for antiviral defense (CRISPR-Cascade or CRISPR-Csy) described herein are disclosed. In some embodiments, the expression codon is optimized to express one or more engineered, non-naturally occurring Type I CRISPR-Cas complex comprising linked subunits described herein. The linked subunits are selected from the group consisting of Cse1, Cse2, Cas7, Cas5, Cas6 (if Cascade) or Csy1, Csy2, Csy3, Csy4 (if Csy), along with crRNA, a nuclease (e.g., Cas3), a NLS, and/or combinations thereof. In some embodiments, the expression codon for a complex containing linked subunits described herein was optimized for expression of an engineered, non-naturally occurring Type I CRISPR-Cas complex in non-native bacterial, archaeal, and eukaryotic systems. In some embodiments, vectors and cells comprised of an engineered, non-naturally occurring Type I CRISPR-Cas complex described herein are provided.

In some embodiments, the expression codon containing a modified Type I CRISPR-Cas complex described herein is provided. In some embodiments, the modified Type I CRISPR-Cas was optimized for expression of an engineered, non-naturally occurring Type I CRISPR-Cas complex include subunits selected from the group consisting of Cse1, Cse2, Cas7, Cas5, Cas6 (if Cascade), or Csy1, Csy2, Csy3, Csy4 (if Csy), along with a modified crRNA, a nuclease (e.g., Cas3), a NLS, and/or combinations thereof. In some embodiments, the modified crRNA includes a modified nucleotide sequence and additional subunits as described herein. In some embodiments, an optimized codon is provided for the expression of an engineered, non-naturally occurring Type I CRISPR-Cas complex in non-native bacterial, archaeal, and eukaryotic systems. In some embodiments, vectors and cells comprised of the engineered, non-naturally occurring extended Type I CRISPR-Cas complex are provided.

Variable crRNA Composition

Two Cse2 subunits form a belly that connects the head and the tail of Cascade, and six Cas7 subunits form a helical backbone. The subunits assemble with crRNA into CRISPR riboncleoprotein complexes that interrogates invading DNA. The Cascade assemblage includes a backbone composed of six Cas7 subunits, which is flanked by a Cas5 tail and a Cas6 head, and the belly composed of two Cse2 subunits, which connects the head and the tail (FIG. 1B). This architecture creates an interwoven ribonucleoprotein structure that kinks the crRNA at 6-nt intervals. In some embodiments, the amino acid at the 6-nt interval is variable. In some embodiments, the amino acid at the 6-nt interval is an indicator.

### Other Type I CRISPR Complexes

Though modified and concatenated systems are illustrated herein in the context of CRISPR-Cascade, also contemplated herein is the use of similarly modified (for instance, concatenated) CRISPR-Csy complexes. It is believed that the bi-directional CRISPR-Cas nucleic acid editing systems described herein are equally applicable for any Type I CRISPR-Cas protein complexes that employ Cas3 for nuclease activity.

The following examples are provided to illustrate certain particular features and/or embodiments. These examples should not be construed to limit the invention to the particular features or embodiments described.

### EXAMPLES

### Example I: Delivery of Complexes to a Target Cell

This Example describes an exemplary method for delivery of two Type I CRISPR-Cas complexes to a target cell targeted bi-directional gene editing using the pair of Type I CRISPR-Cas complexes.

NLS-tagged CRISPR-Cascade complexes (or other Type 1 complexes) designed to target opposite stands of a DNA target are recombinantly expressed and purified using standard methods. Methods for recombinant expression and purification from E. coli have been previously described (Brouns et al., Science 321(5891):960-964, 2008, Jore et al., Nature Structural & Molecular Biology 18:529-536, 2011, Wiedenheft et al., Nature 477:486-489, 2011). NLS-tagged Cas3 is recombinantly expressed and purified separately, or Cas3 is tethered to one of the Cas proteins in the crRNA-guided surveillance complex and thereby co-expressed. For example, Cas3 is fused to Cse1 in Cascade.

A pair of NLS-tagged CRISPR-Cascade or other Type 1 complexes designed to target opposite stands of a DNA target are co-injected (or serially inject) with Cas3 into either the nuclease or the cytoplasm of a eukaryotic cell. Concentrations may be optimized to limit toxicity and off-target effects.

### Example 2: Characterization of Cascade Bi-Directional Gene Editing

This Example describes exemplary methods for characterizing Type I CRISPR bi-directional gene editing of a target sequence in a cell.

In one embodiment, a bi-directional Type I CRISPR gene editing system as described herein is used to target deletion of eGFP. Cells that contain a deletion in eGFP will not be green. These phenotypic changes are observed using fluorescent microscopy or fluorescence activated cell sorting (FACS). Cells that are no longer green will be subjected to DNA sequencing to confirm that the eGFP gene has been modified.

### Example 3: Bi-Directional Gene Editing

This Example describes exemplary use of Type I CRISPR-Cas bi-directional gene editing to modify the genome of a cell, embryo, or organism.

By way of example, a DNA sequence to be modified is selected; the sequence may be in any prokaryotic (e.g., bacteria or archaea) or eukaryotic cell, including animal, plant, fungal, and algal cells. Two target sequences positioned on opposite strands, flanked by PAMs (for the specific Type I CRISPR-Cas complex(es) being used), and separated by about 200-400 bp are suitable for editing.

If the Type I CRISPR-Cas complex(es) are to be delivered as protein complexes, for instance through microinjection into the cell (or its nucleus), the Cas polypeptides required to assemble each complex are produced, purified, and assembled in vitro, to provide the first and second Type I CRISPR-Cas complexes upon assembly with the appropriate guide sequence. The first and second complexes (with associated guide RNA molecules) are introduced into the target cell along with Cas3 nuclease (or a nucleic acid encoding the Cas3 nuclease). Delivery may be directly into the nucleus, or into the cytoplasm of the cell; if the latter, beneficially the complexes and Cas3 nuclease are provided with a NLS attached for subcellular delivery to the nucleus.

Alternatively, if the Type I CRISPR-Cas complex(es) are to be delivered to the cell through expression of constructs encoding their components, the cell is transformed with nucleic acid molecules (such a vector or set of vectors) that encode the polypeptide and RNA components of the complexes, as well as Cas3 (though NLS-tagged Cas3 could be provided as a purified protein even where the complexes are provided from expression cassettes).

Where deletion of the ˜200-400 bp double-stranded DNA sequence is the goal, no further components are required. Innate mechanisms in the cell will repair the double stranded break through prone non-homologous end joining, an error prone process that typically results in insertions or deletions at the site of the dsDNA break. However, in those instances where the intent is to insert a replacement sequence of similar or dissimilar length to the sequence deleted, a repair template is also provided. The repair template or a portion thereof will be “placed” into the DNA molecule through homologous recombination, and thus the characteristics of the repair template are selected to engage the cell's homologous recombination machinery.

This disclosure provides methods, systems, and components for bi-directional Type I CRISPR-Cas gene editing. It will be apparent that the precise details of the methods, systems, and components described may be varied or modified without departing from the spirit of the described invention. I claim all such modifications and variations that fall within the scope and spirit of the claims below.

1. A non-naturally occurring or engineered system for modifying a genomic sequence in a cell, the system comprising:
a first Type I CRISPR-Cas complex comprising:
a first guide RNA having a sequence selected to recognize a first target nucleotide sequence; and
a plurality of Cas polypeptides;
a second Type I CRISPR-Cas complex comprising:
a second guide RNA having a sequence selected to recognize a second target nucleotide sequence; and
a plurality of Cas polypeptides; and
a Cas3 nuclease,
wherein the first and second target nucleotide sequences hybridize to opposite strands of the genomic DNA in the cell at positions that flank the genomic sequence to be modified.

2. The system of claim 1, wherein the first, second, or both the first and second Type I CRISPR-Cas complexes are CRISPR-Cascade complexes, and the plurality of Cas polypeptides comprises Cas6, Cse1, Cse2, Cas7, and Cas5 and/or wherein the first, second, or both the first and second Type I CRISPR-Cas complexes are CRISPR-Csy complexes, and the plurality of Cas polypeptides comprises Csy1, Csy2, Csy3, and Csy4.

3. (canceled)

4. The system of claim 1, wherein the Cas3 nuclease is tethered to a Cas polypeptide in the first or second complex.

5. The system of claim 1, wherein at least two of the Cas polypeptides within a Type I CRISPR-Cas complex are genetically fused.

6. The system of claim 1, further including a repair template.

7. The system of claim 1, wherein the first Type I CRISPR-Cas complex is a CRISPR-Cascade complex and the second Type I CRISPR-Cas complex is CRISPR-Csy complex.

8. A non-naturally occurring cell comprising the system according to claim 1, or a vector or set of vectors expressing components of the system.

9. The cell of claim 8, which is an animal cell, a plant cell, a fungal cell, an algal cell, or a prokaryotic cell.

10. The cell of claim 8, wherein the vector or set of vectors comprises a nucleic acid sequence encoding at least one component of the Type I CRISPR-Cas complex which is codon optimized for expression in bacterial, archaea, or eukaryotic cells.

11. A method for modifying a genomic sequence in a cell, the method comprising:
contacting genomic DNA in the cell with:
a first Type I CRISPR-Cas complex comprising a first guide RNA,
a second Type I CRISPR-Cas complex comprising a second guide RNA, and
a Cas3 nuclease,
wherein the first and second guide RNAs each comprise a sequence that hybridizes to opposite strands of the genomic DNA in the cell at positions that flank the genomic sequence to be modified.

12. The method of claim 11, wherein contacting the genomic DNA with the first and/or second Type I CRISPR-Cas complex and Cas3 comprises:
introducing individual protein or nucleic acid components of the complex or Cas3 directly into the cell;
introducing the Type I CRISPR-Cas complex directly into the cell;
expressing one or more nucleic acids encoding components of the Type I CRISPR-Cas complex or Cas3 in the cell; or
a combination of two or more thereof.

13. The method of claim 11, wherein introducing the Type I CRISPR-Cas complex directly into the cell comprises microinjection into the cell and/or microinjection directly into the nucleus of a eukaryotic cell.

14. (canceled)

15. A method for sequence-specific modification of a target nucleic acid sequence, the method comprising targeting the nucleic acid sequence with the system of claim 1.

16. A method for treating or preventing a disease in a subject in need of treatment or prevention, comprising administering to the subject the system of claim 1.

17. A method of producing a double-stranded break in a nucleic acid molecule in a cell, comprising:
introducing into the cell a first Type I CRISPR-Cas complex comprising a first crRNA comprising a first target sequence, and a second Type I CRISPR-Cas complex comprising a second crRNA comprising a second target sequence, wherein the first and second target sequences hybridize to opposite strands of genomic DNA in the cell at positions that flank the genomic sequence to be modified.

18. The method of claim 11, wherein the first and second Type I CRISPR complexes are CRISPR-Cascade complexes (Type IE), or CRISPR-Csy complexes (Type IF), or any of the other Type I system that uses Cas3 for target degradation.

19. The method of claim 11, wherein modifying the genomic sequence comprises deleting, inserting, or changing wild type genomic sequence through homologous recombination at the double strand break generated by cleavage by the Cas3 nuclease.

20. (canceled)

21. The system of claim 1, wherein the positions that flank the genomic sequence to be modified are at least about 200 base pairs apart but no more than about 1000 base pairs apart.

22. The system of claim 21, wherein the positions that flank the genomic sequence to be modified are about 200 base pairs to about 400 base pairs apart.

23. The system of claim 1, wherein at least one component of one of the complexes, or the Cas3 nuclease, contains a nuclear localization sequence (NLS).


SEQUENCE LISTING (27 sequences)

SEQ ID NO: 1 - protein, 502 aa, Escherichia coli
MNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 2 - protein, 160 aa, Escherichia coli
MADEIDAMALYRAWQQLDNGSCAQIRRVSEPDELRDIPAFYRLVQPFGWENPRHQQALLRMVFCLSAGKNVIRHQDKKSEQTTGISLGRALANSGRINERRIFQLIRADRTADMVQLRRLLTHAEPVLDWPLMARMLTWWGKRERQQLLEDFVLTTNKNA

SEQ ID NO: 3 - protein, 351 aa, Escherichia coli
MTTFIQLHLLTAYPAANLNRDDTGAPKTVVLGGATRLRVSSQSLKRAWRTSALFEQALAGHIGIRSGRIAREAATILIEKGIEDKKAIEWAAKIADYLGKAKNDKKPKDPLTNAETEQLVHISPAEFDAVKALAHQLAEEKRAPKEEDLSLLRKDRMAVDIAMFGRMLANKPEFNVEAACQVAHAFGVSETIVEDDFFTAVDDLRQASEDAGAGHLGETGFGSALFYTYICIDKDLLVENLGGDEALANQTIRAFTEAALKVSPTGKQNSFASRAYASWALAEKGTDQPRSLAAAFYEPINGTRQLDVAVQRITTLRENMNTVYEQKTECASFDVMNKQGSMKDVLDFICA

SEQ ID NO: 4 - protein, 224 aa, Escherichia coli
MRSYLILRLAGPMQAWGQPTFEGTRPTGRFPTRSGLLGLLGACLGIQRDDTSSLQALSESVQFAVRCDELILDDRRVSVTGLRDYHTVLGAREDYRGLKSHETIQTWREYLCDASFTVALWLTPHATMVISELEKAVLKPRYTPYLGRRSCPLTHPLFLGTCQASDPQKALLNYEPVGGDIYSEESVTGHHLKFTARDEPMITLPRQFASREWYVIKGGMDVSQ

SEQ ID NO: 5 - protein, 199 aa, Escherichia coli
MYLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 6 - protein, 888 aa, Escherichia coli
MEPFKYICHYWGKSSKSLTKGNDIHLLIYHCLDVAAVADCWWDQSVVLQNTFCRNEMLSKQRVKAWLLFFIALHDIGKFDIRFQYKSAESWLKLNPATPSLNGPSTQMCRKFNHGAAGLYWFNQDSLSEQSLGDFFSFFDAAPHPYESWFPWVEAVTGHHGFILHSQDQDKSRWEMPASLASYAAQDKQAREEWISVLEALFLTPAGLSINDIPPDCSSLLAGFCSLADWLGSWTTTNTFLFNEDAPSDINALRTYFQDRQQDASRVLELSGLVSNKRCYEGVHALLDNGYQPRQLQVLVDALPVAPGLTVIEAPTGSGKTETALAYAWKLIDQQIADSVIFALPTQATANAMLTRMEASASHLFSSPNLILAHGNSRFNHLFQSIKSRAITEQGQEEAWVQCCQWLSQSNKKVFLGQIGVCTIDQVLISVLPVKHRFIRGLGIGRSVLIVDEVHAYDTYMNGLLEAVLKAQADVGGSVILLSATLPMKQKQKLLDTYGLHTDPVENNSAYPLINWRGVNGAQRFDLLAHPEQLPPRFSIQPEPICLADMLPDLTMLERMIAAANAGAQVCLICNLVDVAQVCYQRLKELNNTQVDIDLFHARFTLNDRREKENRVISNFGKNGKRNVGRILVATQVVEQSLDVDFDWLITQHCPADLLFQRLGRLHRHHRKYRPAGFEIPVATILLPDGEGYGRHEHIYSNVRVMWRTQQHIEELNGASLFFPDAYRQWLDSIYDDAEMDEPEWVGNGMDKFESAECEKRFKARKVLQWAEEYSLQDNDETILAVTRDGEMSLPLLPYVQTSSGKQLLDGQVYEDLSHEQQYEALALNRVNVPFTWKRSFSEVVDEDGLLWLEGKQNLDGWVWQGNSIVITYTGDEGMTRVIPANPK

SEQ ID NO: 7 - protein, 502 aa, Thermus thermophilus
MGSLEKFNLIDEPWIPVLKGGRVVEVGIGEALLRAHEFARIETPSPLEEAVLHRLLLAVLHRALSGPRCPEDVLDWWRKGGFPQDPIRDYLNRFRDRFFLFHPEAPFLQVADLPEENPLPWSKLLPELASGNNPTLFDHTTEENLPKATYAQAARALLVHQAFAPGGLLRRYGVGSAKDAPVARPALFLPTGQNLLETLLLNLVPYTPEDDAPIWEVPPLRLGDLEGARTKWPLTGRTRVYTWPARGVRLLDEGDGVRFMGYGPGVEPLEATHRDPMVAQRLDAKGNLLVLRLSEERSFWRDFSAMLPRQGGKVAATLEHAENLQGELEDEGLEGRITLRVLGQVSDQAKVLDIRREVYPLPSGLLTPKAEENLEKALKMAEELGQGLKHLAQEVAKAVVGERDRGHGRSPYLEELTKLANSLPLERLYWHALDGAFPRFFARVEEEASLDLWREALRGAALEAWKATRRFLGTGARHLKALAQGEQEFGRLLGELGEEVRT

SEQ ID NO: 8 - protein, 169 aa, Thermus thermophilus
MSPGERFLDWLKRLQGQKAWTAARAAFRRSLAFPPGAYPRAMPYVEPFLAKGDWRQEEREAHYLVAALYALKDGDHQVGRTLARALWEKAQGSASVEKRFLALLEADRDQIAFRLRQAVALVEGGIDFARLLDDLLRWFSPERHVQARWAREYYGAGASEEEKKKEVEA

SEQ ID NO: 9 - protein, 367 aa, Thermus thermophilus
MHILQTVAPSNLNRDDTGSPKDALFGGFRRARISSQAQKRAVRVAFRDWPLLSEEERAVRTKRLVEELLRRLAGVEEEELARRAIENALNALGFGVKEGRTEYLLFLGNRELDALAQGIRENLEALSGEVKGKKKAEVPPELKKALERVLDGGKAVDLALFGRMLADRPELGVDAAAQVAHALSTHKVDREFDFYTAVDDLNPKEDTGAGMMGDVEFYSATLYRYAVVDLDKLVENLQGDKELALKGVLAFLEAFALTLPSGKQNSFAAHNPPLFVAFRAGEGMPRNLATAFEAPLRPKEEVPLSRLSAEALLKEWAKFDRVYGPLEPEWKGALDLTGAEGDGVPRLENLKALKEKAAEAVKVLLGV

SEQ ID NO: 10 - protein, 224 aa, Thermus thermophilus
MPTLLLRLQGPLQSWGTRSRFDHRDTWPYPTKSGVVGLLAAALGRDRREDVSDLAALRMGVRVDRKGVLKVDYQTAQNVLAADLKAKRDVQSWRYFLSDAAFLVGLEGERALLEALHRALKNPRFPLYLGRKSYVPSPPPYLPDGLVEAPLAEALEGYPYLLGGKPPEDLLLALEAEEGRLVYDQPAGPFSERRFAARFVREEVLPKEKVPEGPPAWEVTRVAH

SEQ ID NO: 11 - protein, 211 aa, Thermus thermophilus
MWLTKLVLNPASRAARRDLANPYEMHRTLSKAVSRALEEGRERLLWRLEPARGLEPPVVLVQTLTEPDWSVLDEGYAQVFPPKPFHPALKPGQRLRFRLRANPAKRLAATGKRVALKTPAEKVAWLERRLEEGGFRLLEGERGPWVQILQDTFLEVRRKKDGEEAGKLLQVQAVLFEGRLEVVDPERALATLRRGVGPGKALGLGLLSVAP

SEQ ID NO: 12 - protein, 817 aa, Thermus thermophilus
MGEGIGRVFFLKDEVGFQPLSNHQGLVVKLLESWREGDEPLALSPKTKERLLLAARYHDDGKRFTFRIVPDGKGGLTYSFRGHRFRVAQAVQDPYAQALIRGHHDYSTRGVVNLAADFLEEGLGHRFPEDLFLLMMADQLEAELAVRLWQGRAGEVRPFVEFDLLPDGEGGFLLDPWPFRVDEVALDFLVYFHPYRGEEAEVVEGWGRALVGALEEGKVPEAFQEETRRVRLRPWAAEARKADDPEAFYARFGLEPTPFQREVFKVAEGDPAHLLLAPTGTGKTEAAAFPALARGERLVFVLPTRSLVDDLEGRFRRYLKILAQEEGRPKALVVDTGHRQARFRFSPDGQEEATKERHLYHADVILTTLDKLLYRYFGYAEGVKSYTFPRRIHDRRTLFVFDEVHLYEATAWVNFRHLIASLYKAGVRFLVMSATMPGTYREELLLEGTLEHPPARRPSRVLRYMPQGNPMEIIQSHRGKRVLVVLEEVKEAAELYKRLKGEGVFLYHGRLAEGQRRRVFRKVKRRDKAQKPYLLITTPAIEVGVDLDAEVLVTTLCPPENLLQRLGRVNRRGRGQGEAYVVGETYPDYLGSLPEGYLELLKALDGQDLAQGEEERLRQAIRYPKYLDPRAETFFEALQDYVYGLDLTQEPLHRKGFVATRGWTPSVRLRQGEDEVEVPIDRLVGRKEELTPVRVVERLLTDGEKGNRRWEEVPLRSGELYGRELVVDYPYPYDAELGFVELPKVFQRLRHPDPQRVQLLYAPEKDAGEAPEGVMDTDQVTGGGRRVLWYLGESAWAEPAKSGEVAEETEEEEEEGD

SEQ ID NO: 13 - DNA, 1305 bp, Pseudomonas aeruginosa
atgacctctcccctcccaacgcctacgtggcaggagcttcgccagttcatcgaatccttcatccaggagcgcctccagggcaagctggacaaactccaacccgacgaagacgacaagcgccagacattgctggccacccaccggcgggaggcctggctggccgatgccgcccggcgggttggccagttgcagttggtgacccacacgctcaagccgatccatcccgacgcccgcggcagcaacctgcacagcctgccgcaagcacccggccaaccgggcctcgccggttcccatgagctaggtgacaggctggtcagcgatgtggtgggcaatgccgcggcgctggacgtattcaagtttctcagtctccagtatcagggtaaaaatcttctgaactggctgacagaagacagtgccgaggcattacaggcgctgtccgataacgccgaacaggctcgcgaatggcggcaagcgttcatcggcatcacgaccgtcaaaggcgctcccgcgtcccacagcctggccaagcagttgtactttcccctgcccggttccggctaccacctgctagcaccgctgtttcccaccagtctggtgcatcacgtccacgctctgctccgcgaagcgcgcttcggcgacgcggccaaggcggcacgcgaagcgcgcagccgccaggagtcatggccccacggattcagcgagtaccccaacctggcgatccagaagttcggcggtaccaagccgcagaacatcagtcagttgaacaacgagcgccgtggcgagaactggttgctgccatcgcttccgccgaactggcaaaggcagaatgtaaacgcgccgatgcggcactcttcggtcttcgagcatgacttcggaagaactcctgaagtatcccgcctaacccgtaccctgcagcgttttcttgccaagacagtccataacaacctcgccatccgccagcggcgtgcccaattggtggcgcaaatctgcgacgaagccctgcaatacgccgcccgtctgcgcgaactggagcccggctggagcgcaacccccgggtgccaactgcatgacgcggagcagctctggctcgatccgttgcgcgcacagaccgatgagacgttcttgcagcgccgactacgaggtgactggcctgccgaggtcggaaatcgcttcgccaactggctgaaccgggcggtcagcagcgacagtcagatactgggtagcccggaagccgcccaatggagccaggagctgagcaaggaactgacgatgttcaaggagatactcgaagatgagcgtgactga

SEQ ID NO: 14 - protein, 434 aa, Pseudomonas aeruginosa
MTSPLPTPTWQELRQFIESFIQERLQGKLDKLQPDEDDKRQTLLATHRREAWLADAARRVGQLQLVTHTLKPIHPDARGSNLHSLPQAPGQPGLAGSHELGDRLVSDVVGNAAALDVFKFLSLQYQGKNLLNWLTEDSAEALQALSDNAEQAREWRQAFIGITTVKGAPASHSLAKQLYFPLPGSGYHLLAPLFPTSLVHHVHALLREARFGDAAKAAREARSRQESWPHGFSEYPNLAIQKFGGTKPQNISQLNNERRGENWLLPSLPPNWQRQNVNAPMRHSSVFEHDFGRTPEVSRLTRTLQRFLAKTVHNNLAIRQRRAQLVAQICDEALQYAARLRELEPGWSATPGCQLHDAEQLWLDPLRAQTDETFLQRRLRGDWPAEVGNRFANWLNRAVSSDSQILGSPEAAQWSQELSKELTMFKEILEDERD

SEQ ID NO: 15 - DNA, 984 bp, Pseudomonas aeruginosa
atgagcgtgactgatcccgaggcgctgctgttgctgccacgcctgtccatccagaacgccaacgccatttccagccccttgacctggggtttcccttcgcccggcgctttcaccggattcgtccatgccttgcagcgtcgagtcggaatctccctcgatatcgaactggacggtgtcggcatcgtctgtcaccgcttcgaagcgcagatatcgcaaccggccggcaagcgcaccaaggtcttcaacctgacccgcaatccgctgaaccgcgacggctccaccgcagccatcgtcgaagaaggtcgcgcccatctggaggtcagtctgctgctcggagtgcatggcgatggtctggacgatcaccccgcacaggaaatcgccaggcaggtacaggagcaggctggcgccatgcgcctcgccggtggcagcatcctgccctggtgcaatgagcgcttcccggctccgaacgccgaactgctgatgctgggtggcagcgacgaacagcggcgcaagaaccagcgccggcttacccgccgcctgctccccggcttcgccctggtgagtcgcgaggcgttgttgcaacaacatctggaaaccttgcgcaccacgctccccgaagccaccacgctcgatgcactgctcgatctttgtcgaatcaacttcgagcctcctgcaacctcgtccgaagaagaagcatcgccgcccgatgcagcctggcaagtgcgcgacaagcccggctggctggtgccgatcccggctggctacaacgccctgtcacccctgtacctccccggcgaagtgcgaaatgcccgtgatagagaaacaccgctgcgcttcgtcgaaaacctcttcggtctgggcgaatggctcagcccccatcgcgtcgccgcgttatcggacctgctttggtatcaccacgccgagcctgacaaagggctctaccgctggagcacaccccgtttcgtcgaacacgccatcgcataa

SEQ ID NO: 16 - protein, 327 aa, Pseudomonas aeruginosa
MSVTDPEALLLLPRLSIQNANAISSPLTWGFPSPGAFTGFVHALQRRVGISLDIELDGVGIVCHRFEAQISQPAGKRTKVFNLTRNPLNRDGSTAAIVEEGRAHLEVSLLLGVHGDGLDDHPAQEIARQVQEQAGAMRLAGGSILPWCNERFPAPNAELLMLGGSDEQRRKNQRRLTRRLLPGFALVSREALLQQHLETLRTTLPEATTLDALLDLCRINFEPPATSSEEEASPPDAAWQVRDKPGWLVPIPAGYNALSPLYLPGEVRNARDRETPLRFVENLFGLGEWLSPHRVAALSDLLWYHHAEPDKGLYRWSTPRFVEHAIA

SEQ ID NO: 17 - DNA, 1029 bp, Pseudomonas aeruginosa
atgtccaagccaatactgagcaccgcttccgtcctcgccttcgaacgtaagctcgacccttccgacgcccttatgagcgctggcgcctgggcgcaacgcgacgcctcgcaggaatggccggccgtgaccgtgcgcgagaagtccgtacgcggcaccatctccaaccgcctcaagaccaaggaccgtgacccggccaagctggacgcctcgatccagtcgcccaacctgcagacggtggacgtggccaacctgccgagcgacgccgacaccctcaaggtccgcttcaccctccgtgtgctcggcggcgccggaacaccgtctgcctgcaacgacgcggcctaccgtgacaagctgctgcaaacggtcgccacctacgtgaacgatcagggcttcgccgagctggctcgtcgttatgcgcacaacctggccaacgcccgcttcctgtggcgcaaccgggtgggcgcggaagcggtggaagtccgtatcaaccatatccgccagggcgaggtggctcgcgcctggcgcttcgacgccctggccatcggcttgcgcgacttcaaggccgacgccgaactggacgcgctcgccgaactgatcgccagcggtctctcaggcagtgggcatgtcctgctcgaagtggtcgccttcgcccgtatcggcgacggccaggaagtcttcccctcccaggaactgatcctcgacaaaggcgacaagaaaggccagaagagcaagaccctgtacagcgtacgcgatgccgcggccatccactcgcagaagatcggcaatgccctgcgcaccatcgatacgtggtatcccgacgaagatggcctcggccccatcgccgtggagccctacggctccgtcacatcccagggcaaagcctatcgccagcccaagcagaagctggacttctatacgctgctcgacaactgggtactgcgcgacgaggcgcccgccgtggagcaacagcattatgtgatcgccaacctgatccgtggcggcgtgttcggtgaagccgaagagaagtaa

SEQ ID NO: 18 - protein, 342 aa, Pseudomonas aeruginosa
MSKPILSTASVLAFERKLDPSDALMSAGAWAQRDASQEWPAVTVREKSVRGTISNRLKTKDRDPAKLDASIQSPNLQTVDVANLPSDADTLKVRFTLRVLGGAGTPSACNDAAYRDKLLQTVATYVNDQGFAELARRYAHNLANARFLWRNRVGAEAVEVRINHIRQGEVARAWRFDALAIGLRDFKADAELDALAELIASGLSGSGHVLLEVVAFARIGDGQEVFPSQELILDKGDKKGQKSKTLYSVRDAAAIHSQKIGNALRTIDTWYPDEDGLGPIAVEPYGSVTSQGKAYRQPKQKLDFYTLLDNWVLRDEAPAVEQQHYVIANLIRGGVFGEAEEK

SEQ ID NO: 19 - DNA, 564 bp, Pseudomonas aeruginosa
atggaccactacctcgacattcgcttgcgaccggacccggaatttcccccggcgcaactcatgagcgtgctcttcggcaagctccaccaggccctggtggcacagggcggggacaggatcggcgtgagcttccccgacctcgacgaaagccgctcccggctgggcgagcgcctgcgcattcatgcctcggcggacgaccttcgtgccctgctcgcccggccctggctggaagggttgcgggaccatctgcaattcggagaaccggcagtcgtgcctcaccccacaccgtaccgtcaggtcagtcgggttcaggcgaaaagcaatccggaacgcctgcggcggcggctcatgcgccggcacgatctgagtgaggaggaggctcggaaacgcattcccgatacggtcgcgagagccttggacctgcccttcgtcacgctacgcagccagagcaccggacagcacttccgtctcttcatccgccacgggccgttgcaggtgacggcagaggaaggaggattcacctgttacgggttgagcaaaggaggtttcgttccctggttctga

SEQ ID NO: 20 - protein, 392 aa, Pseudomonas aeruginosa
MDHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWFMKSSHHHHHHENLYFQSNADHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 21 - DNA, 3231 bp, Pseudomonas aeruginosa
atgaacatcctgctggtgtcgcaatgcgaaaagcgcgccctgagcgaaacccgccgcattctcgaccagttcgccgagcgccgcggcgaacggacctggcaaacgcccatcactcaagccggactggataccctgcgacgcctgctgaagaaaagcgcacggcgcaacaccgccgtagcctgtcactggatccgcggccgcgaccacagcgaactgctgtggatcgtcggtgatgccagccgcttcaacgcccagggtgcggtgccgaccaacaggacctgccgcgacatcctgcgcaaggaagacgagaacgactggcacagcgccgaggacatccgcctgctgacggtgatggcagcgctgttccacgatatcggcaaggccagccaggccttccaggccaaactgcggaaccgcggcaaaccgatggccgatgcctatcgtcacgaatgggtatcactgcgcctgttcgaagccttcgttggcccaggcagcagcgacgaggactggctgaggcgcctggcggacaagcgagagacgggcgatgcctggctgtcgcaactggccagggacgaccggcaatccgcgccacccggcccgttccagaaaagccggctaccgccgctcgcccaggcggtcggctggttgatcgtcagccatcatcgcctgcccaacggggaccatcgcggcagcgcctcgctggcacgcttgccggcccccatccagagccaatggtgcggcgcacgcgacgcagacgcaaaagaaaaggccgcctgctggcagttcccccacggcctgcccttcgccagcgcccattggcgcgccaggacagcgctatgcgcgcagagcatgctcgagcgtcccggcctgctggctcggggaccggccttgttgcatgattcctacgtcatgcatgtgtcccgactgatcctgatgctcgcggaccaccactattccagtctccctgccgactcccggctgggcgacccgaacttccccttgcacgccaacaccgaccgggacagcggcaaactaaagcagcgcctggacgaacacctgctcggcgtcgccctgcacagtcgcaagctcgccggcaccctgccacgcctggagcgacaactaccgcgccttgcccggcacaagggcttcacccgccgggtcgagcagccgcgcttccgctggcaggacaaggcctacgactgcgcgatggcctgccgcgagcaggctatggagcatggattcttcggcctcaacctggcgtcgaccggttgcggtaagaccctcgccaacggccgtatcctgtatgcgctggccgatccgcaacgcggcgcgcgtttcagcatcgctctcggcctgcgcagcttgaccctgcaaaccgggcaggcctaccgcgagcggctcggcctgggcgacgacgacctcgctatcctggtcggcggcagcgccgcccgcgaactgttcgaaaagcagcaggagcgcctggagcgcagcggtagcgagtcagcccaggagctgctggcggaaaacagccatgtacacttcgccggcacgctcgaggacggccctctacgcgagtggctcggcaggaacagcgcgggaaaccgcctactccaggcgcccatcctggcctgcaccatcgaccacctgatgcccgccagcgaaagcctgcgcggcggacaccagatagcgccactgctccgcctgatgacttccgacctggtgctcgacgaggtcgacgacttcgatatcgacgacctgcccgccctgtcgcggctggtgcactgggccggcctgttcggcagccgcgtgctgctctcctccgcgaccctgccgccggccttggtgcagggcctgttcgaggcctatcgcagcggccgggaaatcttccagcgccatcgtggcgctcccggacgcgctacggaaatccgctgtgcctggttcgacgagttctccagccaatccagcgcccacggcgccgtaacctccttcagcgaagcgcatgcgaccttcgtcgcccagcgtctggcgaagctcgagcaactgccgccacgtcgccaggcgcagctatgcaccgtgcatgccgctggcgaggcccgtcccgcgctgtgccgcgagttggccgggcagatgaatacctggatggctgacctgcatcgctgccatcacaccgaacaccaaggacgtcgcatcagtttcggcctgctacggctggccaacatcgaacccctgatcgaactggcccaggccatcctcgcccagggtgcgcccgaggggttgcatgtccatctgtgtgtctaccattcgcggcatccccttctggtccgctcggccatcgagcgacaactcgatgaactgctgaagcgttcggacgacgacgccgccgcgctgttcgctcgtccgacgctggccaaggcgctccaggccagcacggagcgggatcatctgttcgtcgtactcgcctcgccggtggcggaggtcggtcgcgaccacgattacgactgggccatcgtcgaaccctcctccatgcgctcgatcatccagttggccgggcgaatccgccgccatcgctccggcttcagcggcgaggccaacctatacctgctatcgcgcaatatccgctcgctggaagggcagaatccggcgttccagcggcccggcttcgagacccccgacttccctcttgacagccacgacctgcacgacctgctcgaccccgccctactcgcccgcatcgacgccagcccacgaatcgtcgaaccgttcccactgttcccacgcagccggttggtcgacctggaacaccgacgcctgcgcgcgctgatgcttgccgacgacccaccgtcgtccctgctcggcgtaccgctctggtggcaaaccccggcatcgctcagcggcgccctgcaaaccagccaaccatttcgcgcaggcgccaaggagcgatgctacgccctgctgccggacgaggacgacgaggagcgcttgcatttcagccgctacgaagaagggacctggagcaaccaggacaacctgttgcgcaacctcgacctcacctatggcccgcgcatccagacatggggcacggtcaactatcgggaggagctagtcgcaatggccggccgcgaggacctcgacctgcgtcaatgcgccatgcgctacggcgaggtgagattgcgagaaaacacccagggatggagctaccacccttatttggggttcaagaaatacaactga

SEQ ID NO: 22 - protein, 1076 aa, Pseudomonas aeruginosa
MNILLVSQCEKRALSETRRILDQFAERRGERTWQTPITQAGLDTLRRLLKKSARRNTAVACHWIRGRDHSELLWIVGDASRFNAQGAVPTNRTCRDILRKEDENDWHSAEDIRLLTVMAALFHDIGKASQAFQAKLRNRGKPMADAYRHEWVSLRLFEAFVGPGSSDEDWLRRLADKRETGDAWLSQLARDDRQSAPPGPFQKSRLPPLAQAVGWLIVSHHRLPNGDHRGSASLARLPAPIQSQWCGARDADAKEKAACWQFPHGLPFASAHWRARTALCAQSMLERPGLLARGPALLHDSYVMHVSRLILMLADHHYSSLPADSRLGDPNFPLHANTDRDSGKLKQRLDEHLLGVALHSRKLAGTLPRLERQLPRLARHKGFTRRVEQPRFRWQDKAYDCAMACREQAMEHGFFGLNLASTGCGKTLANGRILYALADPQRGARFSIALGLRSLTLQTGQAYRERLGLGDDDLAILVGGSAARELFEKQQERLERSGSESAQELLAENSHVHFAGTLEDGPLREWLGRNSAGNRLLQAPILACTIDHLMPASESLRGGHQIAPLLRLMTSDLVLDEVDDFDIDDLPALSRLVHWAGLFGSRVLLSSATLPPALVQGLFEAYRSGREIFQRHRGAPGRATEIRCAWFDEFSSQSSAHGAVTSFSEAHATFVAQRLAKLEQLPPRRQAQLCTVHAAGEARPALCRELAGQMNTWMADLHRCHHTEHQGRRISFGLLRLANIEPLIELAQAILAQGAPEGLHVHLCVYHSRHPLLVRSAIERQLDELLKRSDDDAAALFARPTLAKALQASTERDHLFVVLASPVAEVGRDHDYDWAIVEPSSMRSIIQLAGRIRRHRSGFSGEANLYLLSRNIRSLEGQNPAFQRPGFETPDFPLDSHDLHDLLDPALLARIDASPRIVEPFPLFPRSRLVDLEHRRLRALMLADDPPSSLLGVPLWWQTPASLSGALQTSQPFRAGAKERCYALLPDEDDEERLHFSRYEEGTWSNQDNLLRNLDLTYGPRIQTWGTVNYREELVAMAGREDLDLRQCAMRYGEVRLRENTQGWSYHPYLGFKKYN

SEQ ID NO: 23 - DNA, 3234 bp, Pseudomonas aeruginosa PA14
atgaacatcctgctggtgtcgcaatgcgaaaagcgcgccctgagcgaaacccgccgcattctcgaccagttcgccgagcgccgcggcgaacggacctggcaaacgcccatcactcaagccggactggataccctgcgacgcctgctgaagaaaagcgcacggcgcaacaccgccgtagcctgtcactggatccgcggccgcgaccacagcgaactgctgtggatcgtcggtgatgccagccgcttcaacgcccagggtgcggtgccgaccaacaggacctgccgcgacatcctgcgcaaggaagacgagaacgactggcacagcgccgaggacatccgcctgctgacggtgatggcagcgctgttccacgatatcggcaaggccagccaggccttccaggccaaactgcggaaccgcggcaaaccgatggccgatgcctatcgtcacgaatgggtatcactgcgcctgttcgaagccttcgttggcccaggcagcagcgacgaggactggctgaggcgcctggcggacaagcgagagacgggcgatgcctggctgtcgcaactggccagggacgaccggcaatccgcgccacccggcccgttccagaaaagccggctaccgccgctcgcccaggcggtcggctggttgatcgtcagccatcatcgcctgcccaacggggaccatcgcggcagcgcctcgctggcacgcttgccggcccccatccagagccaatggtgcggcgcacgcgacgcagacgcaaaagaaaaggccgcctgctggcagttcccccacggcctgcccttcgccagcgcccattggcgcgccaggacagcgctatgcgcgcagagcatgctcgagcgtcccggcctgctggctcggggaccggccttgttgcatgattcctacgtcatgcatgtgtcccgactgatcctgatgctcgcggaccaccactattccagtctccctgccgactcccggctgggcgacccgaacttccccttgcacgccaacaccgaccgggacagcggcaaactaaagcagcgcctggacgaacacctgctcggcgtcgccctgcacagtcgcaagctcgccggcaccctgccacgcctggagcgacaactaccgcgccttgcccggcacaagggcttcacccgccgggtcgagcagccgcgcttccgctggcaggacaaggcctacgactgcgcgatggcctgccgcgagcaggctatggagcatggattcttcggcctcaacctggcgtcgaccggttgcggtaagaccctcgccaacggccgtatcctgtatgcgctggccgatccgcaacgcggcgcgcgtttcagcatcgctctcggcctgcgcagcttgaccctgcaaaccgggcaggcctaccgcgagcggctcggcctgggcgacgacgacctcgctatcctggtcggcggcagcgccgcccgcgaactgttcgaaaagcagcaggagcgcctggagcgcagcggtagcgagtcagcccaggagctgctggcggaaaacagccatgtacacttcgccggcacgctcgaggacggccctctacgcgagtggctcggcaggaacagcgcgggaaaccgcctactccaggcgcccatcctggcctgcaccatcgaccacctgatgcccgccagcgaaagcctgcgcggcggacaccagatagcgccactgctccgcctgatgacttccgacctggtgctcgacgaggtcgacgacttcgatatcgacgacctgcccgccctgtcgcggctggtgcactgggccggcctgttcggcagccgcgtgctgctctcctccgcgaccctgccgccggccttggtgcagggcctgttcgaggcctatcgcagcggccgggaaatcttccagcgccatcgtggcgctcccggacgcgctacggaaatccgctgtgcctggttcgacgagttctccagccaatccagcgcccacggcgccgtaacctccttcagcgaagcgcatgcgaccttcgtcgcccagcgtctggcgaagctcgagcaactgccgccacgtcgccaggcgcagctatgcaccgtgcatgccgctggcgaggcccgtcccgcgctgtgccgcgagttggccgggcagatgaatacctggatggctgacctgcatcgctgccatcacaccgaacaccaaggacgtcgcatcagtttcggcctgctacggctggccaacatcgaacccctgatcgaactggcccaggccatcctcgcccagggtgcgcccgaggggttgcatgtccatctgtgtgtctaccattcgcggcatccccttctggtccgctcggccatcgagcgacaactcgatgaactgctgaagcgttcggacgacgacgccgccgcgctgttcgctcgtccgacgctggccaaggcgctccaggccagcacggagcgggatcatctgttcgtcgtactcgcctcgccggtggcggaggtcggtcgcgaccacgattacgactgggccatcgtcgaaccctcctccatgcgctcgatcatccagttggccgggcgaatccgccgccatcgctccggcttcagcggcgaggccaacctatacctgctatcgcgcaatatccgctcgctggaagggcagaatccggcgttccagcggcccggcttcgagacccccgacttccctcttgacagccacgacctgcacgacctgctcgaccccgccctactcgcccgcatcgacgccagcccacgaatcgtcgaaccgttcccactgttcccacgcagccggttggtcgacctggaacaccgacgcctgcgcgcgctgatgcttgccgacgacccaccgtcgtccctgctcggcgtaccgctctggtggcaaaccccggcatcgctcagcggcgccctgcaaaccagccaaccatttcgcgcaggcgccaaggagcgatgctacgccctgctgccggacgaggacgacgaggagcgcttgcatttcagccgctacgaagaagggacctggagcaaccaggacaacctgttgcgcaacctcgacctcacctatggcccgcgcatccagacatggggcacggtcaactatcgggaggagctagtcgcaatggccggccgcgaggacctcgacctgcgtcaatgcgccatgcgctacggcgaggtgagattgcgagaaaacacccagggatggagctaccacccttatttggggttcaagaaatacaactgataa

SEQ ID NO: 24 - protein, 324 aa, Pseudomonas aeruginosa
MDDISPSELKTILHSKRANLYYLQHCRVLVNGGRVEYVTDEGRHSHYWNIPIANTTSLLLGTGTSITQAAMRELARAGVLVGFCGGGGTPLFSANEVDVEVSWLTPQSEYRPTEYLQRWVGFWFDEEKRLVAARHFQRARLERIRHSWLEDRVLRDAGFAVDATALAVAVEDSARALEQAPNHEHLLTEEARLSKRLFKLAAQATRYGEFVRAKRGSGGDPANRFLDHGNYLAYGLAATATWVLGIPHGLAVLHGKTRRGGLVFDVADLIKDSLILPQAFLSAMRGDEEQDFRQACLDNLSRAQALDFMIDTLKDVAQRSTVSA

SEQ ID NO: 25 - RNA, 60 nt, Artificial Sequence
  Csy crRNA
cuaagaaauucacggcgggcuugauguccgcgucuaccugguucacugccguauaggcag

SEQ ID NO: 26 - DNA, 50 bp, Artificial Sequence
  Csy DNA target strand
  misc_feature (42)..(43): n is any nucleic acid
aaccttgggcaggtagacgcggacatcaagcccgccgtgaanntcttagg

SEQ ID NO: 27 - DNA, 50 bp, Artificial Sequence
  csy DNA target strand
  misc_feature (8)..(9): n is any nucleic acid
cctaagannttcacggcgggcttgatgtccgcgtctacctgcccaaggtt